(19) United States
(12) Patent Application Publication
(10) Pub. No.: US 2010/0159007 A1
(43) Pub. Date: Jun. 24, 2010

(54) PHARMACEUTICAL COMPOSITIONS FOR TRANSMUCOSAL DELIVERY OF A THERAPEUTICALLY ACTIVE AGENT ON THE BASIS OF SUBMICRON PARTICLES

(75) Inventor: John Nicholas Staniforth, Bath (GB)

Correspondence Address: Davidson, Davidson & Kappel, LLC, 485 7th Avenue, 14th Floor, New York, NY, 10018, US

(73) Assignee: PHARMAKODEX LIMITED, Bath (GB)

(21) Appl. No.: 12/520,417

(22) PCT Filed: Dec. 19, 2007
(86) PCT No.: PCT/GB2007/050766

§ 371 Date: Jan. 25, 2010

(30) Foreign Application Priority Data
Dec. 19, 2006 (GB) 0625322.3

(51) Int. Cl. A61K 9/14; A61K 31/404

(52) U.S. Cl. 424/484; 424/489; 514/415

(57) ABSTRACT

The present invention relates to improved compositions for transmucosal administration, the compositions enabling rapid and efficient uptake of a therapeutically active agent to provide a rapid, effectively durable, predictable and consistent therapeutic effect. In particular, the compositions are intended for buccal and/or sublingual delivery. The invention is particularly suitable for administering therapeutically active agents which have an effect on the central nervous system and even more particularly where rapid onset of this effect is desired or beneficial. The invention is also particularly suitable for administering active agents in low solubility base or acid forms.

The present invention relates to improved compositions for transmucosal administration, the compositions enabling rapid and efficient uptake of a therapeutically active agent to provide a rapid, effectively durable, predictable and consistent therapeutic effect. In particular, the compositions are intended for buccal and/or sublingual delivery of the active agent. The invention is particularly suitable for administering therapeutically active agents which have an effect on the central nervous system and even more particularly where rapid onset of this effect is desired or beneficial. The invention is also particularly suitable for administering active agents in low solubility base or acid forms.

Whilst the pharmacologically active form of many drugs is the base chemical form, or in a smaller number of cases the acid chemical form, it is uncommon for these chemical forms to be administered to mammals, including human mammals, via the peroral route, due to the low and often variable solubility of these chemical forms of the active agent in the fluid of the gastro-intestinal (GI) tract. The lower and potentially variable solubility characteristics of many base and certain acid chemical forms of active agents in GI fluid has meant that pharmaceutical products are instead developed including a salt form or sometimes an ester form of these active agents. For example, less soluble base forms of active agents are frequently converted into a more soluble hydrochloride salt form for improved aqueous solubility and/or solution rate, and/or reduced solubility variability in order to improve pharmacokinetic or other bioavailability parameters following peroral administration of a medicament containing the active agent. In the case of poorly soluble acid forms of drugs, these may be converted, for example, into the sodium salt of the acid chemical form in order to improve aqueous solubility and/or solution rate, and/or reduced solubility variability following peroral administration of a medicament containing the active agent. However, once absorbed into the bloodstream of a patient, dissociation of the free base or acid chemical form of the drug must usually occur as a precursor to pharmacological activity. In cases where rapid onset and/or central (CNS) therapeutic action is desired, the ability to deliver the immediately pharmacologically active base, or sometimes acid, chemical form of the drug into the bloodstream and where appropriate the cerebrospinal fluid, would be advantageous if the problem of poor solubility leading to poor and/or variable peroral drug absorption could be overcome. In consequence, many drugs have never been administered to humans, have never been administered via the peroral route or have never been manufactured, registered or sold as medicines or peroral medicines except in a salt form.

Whilst peroral administration leading to absorption via the gastrointestinal tract is currently the most common route of drug delivery, especially for active agents administered in a salt or ester chemical form, there are a number of drawbacks associated with this type of administration and there are various circumstances where it is less than ideal.

GI administration of pharmaceutically active agents is affected by the “food effects” which contributes to variability in the pharmacokinetic absorption and in pharmacodynamics which has an impact on the efficacy of the absorbed active agent.

GI administration of pharmaceutical compositions may also be adversely affected by GI disturbances (including nausea and vomiting). These conditions (which may be related to the condition to be treated by the pharmaceutical composition, or may actually be caused by the composition being administered) lead to uncertainty as to the dose delivered, as well as variable absorption and efficacy of the dose that is delivered.

Upon administration of a pharmaceutical composition to the GI tract, the composition and the active agent contained therein will be exposed to acids and enzymes which can cause degradation of the active agent and therefore result in variable and reduced drug efficacy.

Administration of a therapeutically active agent via the GI tract may also be adversely affected by efflux and/or metabolism as the active agent crosses the GI mucosa or in the liver (entero-hepatic metabolism). This can lead to abnormally low, or otherwise poor bioavailability or variable distribution, metabolism and/or excretion of the active agent due to effects generally referred to as “first-pass” metabolism.

Finally, in some cases, for example where the active agent is subject to active transport across the GI tract, including saturable transport mechanisms, or is a cytochrome P450 or other metabolism inhibitor, one active agent can block absorption or metabolism of the same or another therapeutic agent. This can lead to undesirable and potentially dangerous drug interactions when such drugs are administered to the GI tract

Some or all of these disadvantages associated with oral administration and absorption of the active agent via the GI tract may be overcome by adopting a pre-gastric transmucosal route of delivery. It is well established that the rate of active agent uptake across the buccal, sublingual, oesophageal, pharyngeal, nasal and pulmonary mucosa can be much faster than that observed as a result of administration via the GI tract. Furthermore, where the active agent is able to rapidly transfer into the systemic circulation from these mucosa, especially from the buccal cavity (including the sublingual area), this avoids one or more of “food effects”, entero-hepatic metabolism, active transport across GI tract and/or cytochrome-mediated metabolism resulting from transfer across GI tract, GI disturbances (including reduced or variable GI motility, absorption, nausea or vomiting) and GI degradation.

As a result, transmucosal administration has the potential to provide drug delivery with great reproducibility, efficiency and rapid onset of action. However, known formulations provided for transmucosal delivery suffer from problems that mean that the therapeutic potential of this route of administration has not yet been fully realised.

Formulations for oral transmucosal delivery via the sublingual or buccal mucosa are known but they often result in the majority of active agent dose being swallowed and thereby being absorbed non-locally in the GI tract, resulting in a slow and variable therapeutic effect. These known formulations are frequently provided in monolithic form, as tablets or lollipops. These will often need to be maintained in contact with the mucosa for an extended period of time which is inconvenient, variable in effect and may be uncomfortable. It also depends upon good cooperation from the patient to ensure that the dose is properly and completely administered. Similarly, oral liquids such as syrups, solutions or suspensions are known for transmucosal administration. In order to encourage dissolved molecules in these liquids to remain in contact with the mucosa, the subject may be instructed to hold the liquid in the mouth for a number of minutes. Use of such a practice is undesirable for a number of reasons. It can be uncomfortable for the subject, especially as the formulations frequently include solvents that sting when held in the mouth for any period of time, are foul-tasting and/or toxic. What is more, relying on the subject to hold the formulation, be it a tablet, lollipop or liquid, in his or her mouth for a period means that the amount of active agent absorbed will be extremely variable, with under-dosing and even overdosing being common. It is also likely that a significant proportion of the active agent will be swallowed.

Fast disintegrating systems such as those from tablets, chewable tablets, wafers and the like have been developed, but these do not provide optimal transmucosal absorption of the active agent because such compositions are usually wetted substantially prior to contact with the oral mucosal surfaces, thereby preventing efficient adhesion of the composition and/or active agent to the mucosa and allowing a significant proportion of the active agent to be swallowed.

Aerosol systems for delivery to and via the mouth are generally limited to relatively low dose drugs and a substantial proportion of the active agent spray does not adhere to or persist at the oral mucosa and is swallowed within a relatively short time after spraying.

In light of the foregoing, it is desirable to provide pharmaceutical compositions which improve transmucosal absorption of the active agent upon administration to a patient. The improvements may be achieved in one or more of the following ways: (i) promotion or enhancement of mucosal adhesion of the composition and/or drug; (ii) promotion or enhancement of persistence of the composition and/or drug at the mucosa, to achieve sufficient flux of the drug into/across the mucosal tissue for the desired therapeutic effect; (iii) promotion or enhancement of spreading of the composition and/or drug across the mucosal area; (iv) promotion or enhancement of transmucosal flux to deliver a sufficient dose of the drug to achieve the desired therapeutic effect either locally or systemically; (v) promotion or enhancement of transmucosal flux to deliver a sufficient dose of the drug to achieve the desired systemic therapeutic effect more rapidly; (vi) promotion or enhancement of transmucosal flux to deliver a sufficient dose of the drug to achieve the desired central (CNS) therapeutic effect more rapidly; and (vii) promotion or enhancement of transmucosal flux to deliver a sufficient dose of the drug to achieve therapeutically effective dosing from the sublingual and/or buccal areas that avoid differences in pharmacokinetic/pharmacodynamic profiles resulting from dosing with or without food and/or that avoid “first-pass” (hepatic) metabolism.

According to a first aspect of the present invention, compositions are provided wherein the compositions comprise submicron particles of a therapeutically active agent and wherein the active agent has poor aqueous solubility characteristics. The compositions according to the present invention may be presented as a solid dosage form. One possible presentation is as a tablet. Preferably, the compositions may be in the form of a free-flowing powder or granulate.

Herein, the term “transmucosal” is used to refer to “pre-gastric” absorption, i.e. absorption which occurs principally in the region above the stomach but after the mouth or nose, across the buccal, sublingual, oesophageal, pharyngeal, nasal and/or pulmonary mucosa.

Although the mucosal lining of the GI tract is preferably not the primary target for transmucosal absorption of the compositions of the present invention, that does not mean that none of the drug is to be absorbed via the GI tract or that a degree of such absorption is undesirable or without therapeutic value. For clarity, drug absorption from the “pre-gastric” region will be particularly important to shortening time taken to achieve maximal drug concentration (tₘₐₓ) whilst subsequent absorption including any from GI tract, may be important to achieving desirable overall pharmacokinetic behaviour as defined by peak dose concentration (Cₘₐₓ) and “total” dose (area under curve or AUC) parameters.

Submicron particles are defined as a collection of particles in which a majority of particles have a diameter of less than 10 μm and preferably less than 1 μm. In preferred embodiments of the present invention, the majority of the submicron particles have a diameter of at least 100 nm and less than 10 μm, and more preferably have a diameter of between 150 nm and 5 μm, between 150 and 999 nm, between 150 and 990 nm or between 150 and 950 nm. Preferably, at least 60%, at least 70%, at least 80%, at least 90% or at least 95% of the submicron particles have a diameter falling within one or more of the abovementioned ranges.

In further embodiments, the mean or median diameter of the submicron particles according to the present invention is between 200 nm and 5 μm, between 300 nm and 2 μm, between 400 and 900 nm, or is approximately 500 nm.

Therapeutic agents that exhibit poor aqueous solubility characteristics are defined herein as being insoluble or sparingly soluble in water. Preferably, the therapeutic agent has a solubility of 1 part (by weight) drug in no less than 30 parts (by volume) water (at 25° C.). In some embodiments, the drug has a solubility of 1 part drug (by weight) in no less than 100 parts water, in no less than 1,000 parts water, in no less than 5,000 parts water, or in no less than 10,000 parts water (by volume) (at 25° C.).

The submicron particles may comprise two or more therapeutic agents (including different forms of the same therapeutic agent). In some embodiments, the submicron particles consist of one or more therapeutic agents (including different forms of the same therapeutic agent).

In many cases, the sparingly soluble or insoluble therapeutic agents will be the base forms of the agents. In conventional pharmaceutical compositions, it is common for the soluble salt forms of agents to be included, as these are more soluble and therefore are released from the dosage form more readily when delivered, for example, via the GI tract.

The reason for using forms of therapeutic agents that are, at best, sparingly soluble in water in the compositions of the present invention is that the wetting and dissolution of the active agent anywhere other than in the micro-environment close to or at the mucosal surface is actually undesirable. In order to optimise transmucosal delivery, the active agent needs to remain in contact with the mucosa, i.e. it needs to remain in a form that adheres to the mucosal surface and is subsequently absorbed transmucosally. This is especially important where absorption is to occur via the buccal or sublingual mucosa.

The mouth of a patient is an aqueous environment and saliva is present in order to assist swallowing of material. However, swallowing the active agent is to be kept to a minimum, especially when transmucosal delivery is sought to overcome a variety of ADME (Absorption, Distribution, Metabolism, Excretion) problems associated with some active agents. The pharmacokinetic profile of an active agent exhibiting one or more of “food effects”, entero-hepatic metabolism, GI disturbances, GI degradation when swallowed will be different from that where an amount of drug is absorbed transmucosally, for example from the buccal or sublingual regions. Whilst the swallowed active agent may not be ultimately lost, as at least some of it may eventually be absorbed from the GI tract, any swallowed active agent will not have the desired rapid effect and its effect is likely to be variable and difficult to predict. If the active agent dissolves readily in water, it is to be expected that, upon introduction into the buccal cavity for buccal or sublingual transmucosal delivery, at least some of the active agent will dissolve in the saliva present and will not be directed to become adhered to or persist in the micro-environment around the oral mucosal surfaces and a substantial portion of the active agent will be swallowed.

In the present invention, it is preferred for the active agent to remain in a substantially undissolved state until positioned in the micro-environment adjacent to the mucosal membrane and it should remain there for long enough for a sufficient amount of the active agent to be absorbed.

In preferred embodiments, the submicron particles of insoluble active agent adhere to the mucosal surfaces and/or persist in the micro-environment close to the mucosal surfaces so as to enable at least 5% of the active agent dose to be absorbed transmucosally. More advantageously, at least 10%, at least 15%, at least 20%, at least 25%, at least 30%, at least 35%, at least 40%, at least 45% or at least 50% of the active agent is absorbed. Absorption of up to 95%, 96%, 97%, 98% or 99% may be observed. In one embodiment approximately 60% of the administered dose of active agent is absorbed or approximately 60% of the metered dose.

In embodiments of the present invention, more than 20% of the dose of active agent should enter the systemic circulation in the head and neck region and not the GI/abdominal region. Preferably, at least 30% of the dose of active agent, at least 40%, at least 50%, at least 60%, at least 70% or at least 80% should enter the systemic circulation in this region.

In embodiments of the present invention, at least about 2% of the dose of active agent should enter the systemic circulation within 15 to 30 minutes following administration. Preferably, at least 5% of the dose of active agent, at least 10%, at least 20%, at least 30%, at least 40%, at least 50%, at least 60%, at least 70, at least 80% or at least 90% should enter the systemic circulation within 15 to 30 minutes following administration.

In some embodiments, it is desirable for the active agents included in the compositions of the present invention which have low aqueous solubility to also have higher lipid solubility. This can enhance transmucosal absorption into the systemic circulation and any desired subsequent absorption into the CNS. Preferably, the lipid solubility of the active agent is sufficiently high to promote rapid mucosal absorption and, where CNS activity is desired, rapid transfer across the blood-brain-barrier and into the brain and brain stem. In some embodiments of the invention, the active agent has a hydrophilic lipophilic balance (HLB) number of less than 15 and preferably less than 10, less than 8 or less than 5. For methods of calculating HLB number values, see (i) Griffin W C: “Classification of Surface-Active Agents by ‘HLB’” Journal of the Society of Cosmetic Chemists 1 (1949): 311, (ii) Griffin W C: “Calculation of HLB Values of Non-Ionic Surfactants” Journal of the Society of Cosmetic Chemists 5 (1954): 259 and (iii) Davies J T: “A quantitative kinetic theory of emulsion type, I. Physical chemistry of the emulsifying agent” Gas/Liquid and Liquid/Liquid Interface. Proceedings of the International Congress of Surface Activity (1957): 426-438).

It is recognised that the base form of many active agents is quickly taken up into the bloodstream and is then able to cross the blood-brain-barrier more readily than the salt forms and so is better at exerting an effect on the central nervous system. Therefore, in a particularly preferred embodiment of the invention, the therapeutic agent included in the composition is in the base form. In another preferred embodiment, the therapeutic agent is in a demethylated form.

Submicron particles of the active agent are used in the compositions of the present invention because they exhibit high surface energy and are therefore inherently “sticky”. When these submicron particles are locally administered to a mucosal membrane, they tend to stick to the mucosal surface and remain adjacent to the mucosa for long enough for the active agent to be transmucosally absorbed.

The submicron particles which may be poorly soluble in water are preferred to individual molecules in solution or fast dissolving particles (such as those from known powders, tablets, chewable tablets, wafers and the like) because the dissolved or dissolving molecules will not be presented in preferentially high concentrations in the micro-environment close to the mucosal surface, nor will they adhere to the mucosa in preferentially high concentrations and a significant proportion of the active agent is likely to be swallowed before it is transmucosally absorbed.

In a preferred embodiment of the invention, solid state formulations of active agents are provided which may be administered sufficiently closely to the mucosa as to enable adhesion of a sufficient amount of the active agent to promote a sufficient and sufficiently rapid local absorption across the mucosa.

The inclusion in the compositions of the present invention of the active agent in fine particle form enables a sufficient mucosal adhesion, persistence and local dissolution in the microenvironment of the mucosa to allow a sufficient amount of material to promote a sufficient and sufficiently rapid local absorption across the mucosa. Preferably, a large proportion of the fine particles of active agent are in the submicron range.

In a preferred embodiment of the invention, the compositions further comprise one or more inert materials. These inert materials are preferably physiologically acceptable. Preferably, the active agent material is treated to produce submicron particles of active agent dispersed in one or more inert material. Preferably, the inert material is provided in the form of particles, within which submicron particles of active agent are dispersed. Submicron active particles may be formed by milling, co-milling, granulation, spray granulation, spray drying, spray congealing, spray evaporation, precipitation, co-precipitation, ultrasonic spraying, supercritical fluid processing or the like, so as to embed submicron active agent particles in one or more particles of the inert material, so that the inert material may be said to act as a matrix.

Where the submicron particles of active agent are dispersed within particles of inert material, the particle of inert material preferably has a diameter which allows easy handling of the particles, i.e. good flowability and the like, as well as sufficiently rapid dissolution to release the submicron particles of active agent in desirable concentrations in the micro-environment close to mucosal surfaces.

In one embodiment, the particles of inert material (including the active agent dispersed therein) have a diameter of between 1 μm and 1000 μm eg between 1 μm and 710 μm. This particle size affords good handleability and allows the particles to be easily and uniformly blended with other particles in powders. Particles with a diameter of at least 10 μm are preferred where these particles are administered to the buccal cavity, as this particle size will minimise risk of accidental inhalation that could lead to deposition in the lung.

Particles with a diameter of less than 800 μm eg less than 500 μm are preferred for reasons of weight and content uniformity, adhesion, solubility characteristics and the like. More preferably, particles with a range between 10 and 600 μm and most preferably particles between 45 and 500 μm.

The size of the submicron particles of active agent embedded within the inert material can be determined by dissolving the inert material and measuring the size of the undissolved active agent. Preferably, the size of these submicron particles of active agent is between 100 nm and 1.5 μm. More preferably, the size range is 200 to 1000 nm, 300 to 900 nm or 400 to 750 nm.

Preferably, the inert material is selected to dissolve or disperse rapidly, so that they release the submicron particles of the active agent dispersed therein upon administration of the composition. Suitable inert materials include those having GRAS (Generally Recognised As Safe), pharmacopoeial and/or regulatory acceptance or acceptability. Examples of suitable inert materials include: water, other aqueous media (e.g. water-ethanol mixtures and isotonic water-glycerol mixtures) or non-aqueous media leading to residual levels in a pharmaceutical product suitable for administration to humans or animals; surfactants, including non-ionic surfactants, anionic, cationic and amphoteric surfactants such as polysorbates (e.g Tweens.), and polyoxyethylene sorbitan fatty acid esters, sorbitan esters (e.g. Spans, sorbitan monostearate), including sorbitan laurate, sorbitan oleate, sorbitan palmitate, sorbitan sesquioleate, sorbitan stearate, sorbitan trioleate, sorbitan tristearate, sucrose esters, poloxamers (e.g. Pluronics) including poloxamer 188, poloxamer 407 and poloxalene, polyoxyl castor oils, polyoxyl hydrogenated castor oils, propylene glycol diacetate, propylene glycol laurate, propylene glycol dilaurate, propylene glycol monopalmitostearate, quillaia, diacetylated monoglycerides, diethylene glycol monopalmitostearate, p-di-isobutyl-phenoxypolyethoxyethanol, ethylene glycol monostearate, self-emulsifying glyceryl monostearate, macrogol cetostearyl ethers, cetomacrogol, polyoxyethylenes, polyethylene glycols, polyoxyl 20 cetostearyl ether, macrogol 15 hydroxystearate, macrogol laurel ethers, laureth 4, lauromacrogol 400, macrogol monomethyl ethers, macrogol oleyl ethers, menfegol, mono- and di-glycerides, nonoxinols, octoxinols, glyceryl distearate, glyceryl monolinoleate, glyceryl mono-oleate, tyloxapol, free fatty acids (e.g. oleic acid, palmitic acid, stearic acid, behenic acid, erucic acid) and their salts and esters (e.g. sodium stearate, magnesium stearate, aluminium monostearate, calcium stearate, zinc stearate, sodium cetostearyl sulphate, sodium oleate, sodium stearyl fumarate, sodium tetradecyl sulphate, soft soap, sulphated castor oil, glyceryl behenate), phospholipids and phospholipid-containing materials, including phosphatidylcholine, lecithin, colfosceril palmitate, phosphatidyl glycerol, Lucinactant, animal lung extracts and modified animal lung extracts; sodium lauryl sulphate and docusate sodium, benzalkonium chloride, cetrimide and nonylphenols, and other emulsifiers (including polymeric materials); soluble small molecules including amino acids (e.g. taurine, aspartame) and especially bioadhesive materials, including sugars, sugar alcohols, dextrates, dextrins, dextrans and hydrating agents, especially urea; and soluble large molecules, especially biodegradable polymers capable of dissolving or dispersing relatively rapidly, including natural and semi-synthetic macromolecules such as phospholipids and especially those that can aid adhesion to and/or spreading across mucosal surfaces (e.g. phosphatidyl choline, lyso-phosphatidyl choline, colfosceril palmitate, phosphatidyl glycerol and mixtures of such materials including with e.g. tyloxapol, cetyl alcohol, free fatty acids), vitamins, natural oils including orange, lemon, bergamot, anise; alcohols, including menthol and cetyl alcohol and cholesterol, natural polymers such as xanthan, guar and alginates, synthetic polymers such as PVP and PVA, semi-synthetic polymers such as cellulose derivatives (e.g. HPMC and HPC) and starch derivatives. Amongst the preferred inert materials are HPMC and mannitol.

Surfactants appear to be important ingredients for optimising the transmucosal absorption of the active agent, by controlling the release of the submicron particles from the matrix upon administration of the compositions according to the invention.

Solvents may be added to the surfactants in the compositions of the invention. Suitable solvents include alcohols and oils (such as menthol, eucalyptol, orange oil, lemon oil, etc.). Co-solvents, such as polyethylene glycols, may also be included. One preferred solvent is menthol.

The surfactants, solvents and other inert ingredients improve the compositions by (i) in the case of emulsions, acting as emulsifying agents in producing submicron material that can be subsequently dried; (ii) in the case of microencapsulation, acting as agents in producing submicron microcapsule material that can be subsequently dried; (iii) in the case of precipitation, by enabling solution and then anti- (or non-) solvent systems to be produced in order to yield submicron particles that can be recovered by drying or recovered by centrifugation, filtration, etc.; and (iv) in the case of preparation of submicron material by milling or co-milling, acting as milling aids to promote more efficient or effective micronisation.

An advantage of providing the submicron particles embedded in an inert particle matrix or a matrix of inert particles is that individual submicron active agent particles may be kept apart from one another and this is desirable in order to prevent cohesive agglomeration. Particles of submicron dimensions will tend to self-agglomerate by cohesion caused by surface free energy effects, forming agglomerates that are 3 to 5 μm in diameter, or even larger. These cohesive agglomerates of active agent particles are undesirable; they have less surface energy than the individual submicron particles and are therefore less likely to adhere to the mucosa. What is more, even if the agglomerates do adhere to the mucosa, relatively few submicron particles will be positioned immediately adjacent the mucosal membrane and so less of the active agent would be expected to be absorbed transmucosally. Furthermore, cohesive agglomerates of active agent particles will have a reduced dissolution rate in the micro-environment close to the mucosal surface.

In some embodiments of the present invention, the compositions further include other materials, preferably in particulate form. Thus, in some embodiments, the compositions comprise submicron particles of active agent, preferably embedded in or with one or more larger particles of one or more inert materials, and particles of a further material. The further material may be included to act as a diluent, especially where the amount of active agent to be administered is small. Alternatively, the further material may be included in order to improve the organoleptic properties of the composition.

In order to be acceptable for reasons of mouthfeel and comfort (taking into account bulk volume, mouth drying effects, saliva generation effects, etc.), the total amount of the composition of the present invention (including both inert and active components) to be administered at any one time should be restricted to masses below a maximum quantity. In order to be acceptable for reasons of accurate dose pre-metering or metering, the total amount of the composition of the present invention (including both inert and active components) to be administered at any one time should be restricted to masses above a minimum quantity. Preferably, the maximum mass for delivery to the buccal cavity should be no more than 3 g. Preferably, the minimum mass should be at least 1 mg. More preferably the delivered powder mass should be between 5 mg and 2 g, between 50 mg and 1.5 g, or should be approximately 1 g. Preferably, the maximum mass for delivery to the sublingual regions should be no more than 1 g, and the minimum mass should be at least 1 mg. More preferably, the sublingually delivered powder mass should be between 5 mg and 500 mg, between 50 mg and 250 mg, or should be approximately 150 mg. The actual most preferred masses within these ranges will depend on various factors such as the size of the dose of the drug, solubility characteristics of the drug, mucosal adhesion and penetration characteristics of the drug, age of patient, therapeutic condition to be treated, ability of patient to generate saliva, etc.

The compositions of the present invention may be provided in the form of a loose powder, a capsule containing a loose or compressed powder, a blister or other unit dose presentation containing a loose or compressed powder, or they may be in the form of a tablet, preferably formed by compressing a powder composition.

In one embodiment of the invention, the composition is for buccal or sublingual administration. The composition is placed in the appropriate part of the buccal cavity and the submicron particles become adhered to the mucosal surfaces and the active agent is subsequently absorbed transmucosally to provide a local or systemic effect. Where the submicron particles of active agent are embedded in one or more larger particles of inert material, the inert material rapidly dissolves once it is wetted in the buccal cavity, thereby releasing individual, largely unagglomerated submicron particles of active agent which adhere to the mucosal surfaces and are absorbed.

In a preferred embodiment, the composition is placed in the buccal cavity in the form of a loose powder. It is clear that a loose powder will be able to spread over the mucosal surfaces, ensuring that more of the active agent comes into direct contact with the mucosa and is therefore ideally placed for absorption. The spread of the powder within the buccal cavity will also improve rapid wetting of the composition and “release” of the submicron particles of active agent.

Powder forms of the compositions according to the present invention will have other benefits. For example, where it is important to ensure that the dose of active agent is administered and not subsequently removed from the buccal cavity, the administration of a powder will make it difficult, if not impossible, to remove the dose of powder once it has been placed in the buccal cavity. Thus, powders are an attractive form in which to administer drugs to treat conditions such as schizophrenia, bipolar disorder and depression, or to treat children.

Other dosage forms are also suitable for delivery to the buccal cavity, provided that they disintegrate and release the submicron particles of active agent rapidly upon being placed in the buccal cavity. Such dosage forms include compressed tablets, capsules, wafers and the like. It may be desirable to include additional components in the composition of the invention in order to ensure rapid disintegration. Suitable disintegrants are known and include starch, cross-linked sodium carboxymethylcellulose (croscarmellose), sodium starch glycollate, cross-linked PVP (crospovidone), gas couples (e.g. carbonate salts and fruit acids) and ion exchange resins. Alternatively, the inert material included in the composition may be selected to provide the desired rapid disintegration and release of the submicron particles.

Where they are to be administered to the nasal mucosa, the compositions according to the present invention are preferably in the form of a loose powder, as this will be most comfortable for the patient. In some cases of pre-gastric administration of active agents it may be necessary or desirable to adjust the tonicity (including osmolarity or osmolality) and/or ionic strength in order to ensure minimal discomfort or irritation at the mucosal surface. Adjustment of these characteristics may be achieved using mineral or organic acids, alkalis and/or salts and/or other buffer agents in appropriate concentrations.

It has been discussed above how the buccal and/or sublingual mucosa are the primary target area for absorption of the active agent upon administration of the compositions according to the present invention to the buccal cavity. The compositions deliver the sparingly soluble active agent in the form of submicron size to reduce the amount of the active agent that is accidentally swallowed. Encouraging buccal and/or sublingual transmucosal absorption in this way ensures rapid onset of the therapeutic action and administration of a consistent and predictable dose. However, it is likely that at least some of the active agent will be swallowed and the swallowed active agent is likely to have a therapeutic effect when it is absorbed via the GI tract.

In some embodiments of the present invention, it is desirable for the composition to provide secondary absorption of the active agent via the GI tract, in addition to the primary absorption via the buccal and/or sublingual mucosa. This secondary absorption can provide a second, delayed therapeutic effect in addition to the initial, rapid effect resulting from the primary absorption. Thus, the compositions according to the invention may provide a rapid onset of therapeutic action, combined with delayed and/or sustained action. The active agents having the immediate and delayed and/or sustained action may be the same or different.

In some embodiments of the present invention, the compositions further include particles comprising an active agent which is to be swallowed and absorbed via the GI tract. These particles may, for example, include a coating to prevent release of the active agent within the buccal cavity, thereby encouraging the active agent to be swallowed and released in the GI tract. Suitable coatings are well known and include ethylcellulose, HPMC, HEC, HPC, CAP and other cellulose ethers and esters, PVP, either alone or together. Another measure that may be taken to encourage GI absorption is to use an aqueous soluble salt form or amorphous form of the active agent or a mixture of salt form or amorphous form with lower solubility base or acid forms. The soluble active agents are more likely to become dissolved in the saliva present in the buccal cavity and to be swallowed.

Methods of producing submicron particles of active agent and/or matrix particles containing such submicron particles dispersed within a relatively rapidly dissolving or dispersing matrix of other, usually inert ingredients having GRAS, pharmacopoeial and or regulatory acceptability or acceptance, include, but are not limited to, emulsification, emulsification followed by solvent evaporation/cross-linking and emulsion polymerization, as well as recovery of submicron particles from active agent dissolved in single phase liquid systems, two-phase liquid systems or multi-phase systems.

The term “relatively rapidly” includes dissolution or dispersion of a matrix (defined as a single larger particle containing submicron drug particles, or submicron drug particles with a multiplicity of inert ingredient particles) at the mucosal surface in a period not exceeding 2 hours. Suitable inert ingredients for mixing with, emulsifying or inclusion in, submicron particle matrices include: water, other aqueous media (e.g. water-ethanol mixtures, isotonic water-glycerol mixtures) or non-aqueous media leading to residual levels in a pharmaceutical product suitable for administration to humans or animals, surfactants, other emulsifiers (including polymeric material); polymers, biodegradable polymers capable of dissolving or dispersing relatively rapidly, bioadhesive materials, including sugars, sugar alcohols, polymers, biodegradable polymers, natural molecules such as urea, phospholipids, such as phosphatidyl choline, and semi-synthetic variants such as colfosceril palmitate, phosphatidyl glycerol, etc., or mixtures of such materials), vitamins, natural oils, alcohols and cholesterol.

Methods of producing solid state material of, or containing, submicron particles include, but are not limited to, the following: preparation of colloids, micelles or other forms of submicron particles of active agent either alone or together with other ingredients such as those listed above by condensation methods; microencapsulation methods; precipitation, including precipitation using aqueous, organic and supercritical fluid methods (for example, DELOS—depressurization of an expanded liquid organic solution, RESS—rapid expansion of supercritical solutions, and GAS—Gas Antisolvent); high pressure homogenisation including colloid milling; other methods of milling including wet milling, dry milling (or micronisation), co-milling, sonic and vibration milling, cryogenic milling; spray methods, including spray drying, spray cooling (prilling), spray fluid bed drying, spin flash drying (tornado/cyclone drying), ultrasonic spray recovery methods including ultrasonic spray drying, electro-spray recovery including electro-spray drying methods, supercritical fluid recovery including SCF spray drying methods; fluidised bed processing methods, including pressure swing methods and freeze drying methods.

Methods and processes that are suitable for preparing compositions according to the present invention, or that may be adapted to prepare such compositions, are disclosed in earlier patent applications published as WO 2004/011537, WO 2005/073296, WO 2005/075546, WO 2005/073300, WO 2005/075547, US 2004/0191324, US 2004/0197417 and US 2004/0253316. This list is not exhaustive.

One preferred process for producing the particles used in the present invention is a spray drying process. The water insoluble active agent is included in a solution, suspension or emulsion together with suitable solvents, surfactants and other inert materials, as discussed above. This mixture is then spray dried to produce particles comprising the active agent embedded in a matrix. When these particles are dissolved, they release particles of the active agent which may be transmucosally absorbed. As the skilled person would appreciate, the size and other properties of the spray dried particles may be controlled by the spray drying parameters and the properties of the solution or suspension being spray dried. More information about suitable spray drying processes is provided in the Examples.

It may be desirable for the spray dried particles to undergo a secondary drying step, in order to adjust the moisture content of the spray dried particles. This is likely to be most relevant where the spray dried particles are particularly sensitive to moisture. When the ambient air has low humidity and/or where the spray drying is conducted using compressed air which is dry, such secondary drying is probably not necessary.

In an alternative preferred process, the particles are produced by a milling step. Milling of the active agent and a surfactant can, for example, result in particles with an average particle size of less than 2 μm (and preferably approximately 1.47 μm). One suitable mill for this purpose is a cryogenic mill. More information about this and other suitable milling processes is provided in the Examples.

The active agent in the submicron particles is preferably in crystalline form as this is more stable. However, some amorphous material may be present in some embodiments, particularly where the active agent does not suffer from stability problems or where the composition may be stored in a way that ingress of moisture is not an issue.

Numerous drugs are attractive candidates for use in the compositions according to the present invention for transmucosal delivery. These drugs may be defined in terms of the following characteristics and the examples given are base forms except where indicated otherwise.

1) Drugs that exhibit high (>25%) “first-pass” metabolism.

Examples of such drugs include acids, bases or salts of sildenafil, tadalafil, vardenafil, clopidogrel (and insoluble bisulphate salt form), levodopa, irbesartan (acid), aripiprazole, aprepitant, metoprolol, propranolol, lidocaine, propafenone, verapamil, nitroglycerin.

2) Drugs that show “food effects”.

These drugs show significant differences in the pharmacokinetic measurements such as tₘₐₓ, Cₘₐₓ or AUC, and/or pharmacodynamic measurements of drug efficacy, when a drug is given in “fasted” versus “fed” conditions. Examples of such drugs include sildenafil and other PDE5 inhibitors such as tadalafil, vardenafil and levodopa, valsartan (acid form), nifedipine, nimodipine, nicardipine, amlodipine, mebeverine, betahistine, atazanavir, indinavir, lopinavir, ritonavir, nelfinavir.

3) Drugs that exhibit variable or poor absorption due to GI disturbances.

The GI disturbances include variable or reduced motility resulting either from the condition to be treated (e.g. migraine and epilepsy) or from the presence of the drug itself in the GI tract, and effects such as nausea and vomiting that are caused by either the condition to be treated (e.g. migraine and motion sickness) or that are drug induced (e.g. caused by chemotherapeutic and pharmacological agents). Examples of such drugs include acids, bases or salts of anti-migraine drugs such as prochlorperazine, amitriptyline, sumatriptan, eletriptan, frovatriptan, almotriptan, zolmitriptan, etc.; loxapine, buspirone, anti-emetic drugs such as ondansetron, aprepitant, etc., proton pump inhibitors such as omeprazole, esomeprazole; moxisylate, naftidofuryl, ephedrine, eroprostenol, fondaparinux, protamine, clopidogrel, dipyridamole, etamsylate, colestipol, ezetimibe, bezafibrate, ciprofibrate, fenofibrate, gemfibrozil, atorvastatin, fluvastatin, pravastatin, rosuvastatin, simvastatin, montelukast, cetirizine, aripiprazide, modafinil, sibutramine, cinnarizine, cyclizine, spironolactone, triamterene, amiloride, furosemide, torasemide, flecainide, procainamide, mexiletine, captopril, cilazapril, enalapril, fosinopril, imidopril, lisinopril, moexipril, perinopril, quinapril, ramipril, trandolapril, telmisartan, lercanidine, nicardipine, nimodipine, verapamil, nicorandil, cilostazol, meclozine, promethazine, chlorpromazine, perphenazine, prochlorperazine, trifluoperazine, domperidone, metoclopramide, dolasetron, granisetron, ondansetron, tropisetron, aprepitant, aprepitant with dexamethasone, aprepitant with budesonide, fluticasone, or other steroids, nabilone, hyoscine, nefopam, ergotamine, methysergide, ethosuximide, gabapentin, levitaracetam, topiramate, valproic acid/valproates, levodopa, co-beneldopa, co-careldopa, amontadine, apomorphine, entacapone, lisuride, pramipexole, ropinirole, selegiline, trihexyphenidyl, riluzole, tetrabenazine, acamprosate, disulfiram, bupropion, nicotine, donepezil, galantamine, riastigmine, fluconazole, griseofulvin, ketoconazole, zalcitabine, aciclovir, famciclovir, valaciclovir, ganciclovir, zanamivir, alendronic acid/alendronates, pamidronic acid, elidronic acid, ibandronic acid, risedronic acid, clodronic acid, tiludronic acid, zoledronic acid, bromocriptine, quinagolide, buserelin, goserelin, leuprorelin, nafarelin, triptorelin, ritodrine, mycophenolate, tacrolimus, famotidine, rabeprazole, pantoprazole, cimetidine, ranitidine, lansoprazole, probenecid, foscarnet, adefovir, oseltamivir, artemether, lumefantrine, chloroquine, mefloquine, primaquine, proguanil, atovaquone, quinine, mepacrine, piperazine, chlorpropamide, glibenclamide, gliclazide, glimepiride, glipizide, gliquidone, tolbutamide, metformin, acarbose, pioglitazone, repaglinide, rosiglitaz one.

4) Drugs that undergo chemical or enzymatic degradation.

This degradation will tend to occur in the stomach (e.g. acid hydrolysis) or in the intestines (e.g. bile acids, mixed esterase attack, etc.).

5) Drugs having a principle site of action in the central nervous system.

These drugs must cross the blood-brain-barrier to access the intended site of action.

6) Drugs that are intended to provide rapid or acute treatment of symptoms.

These drugs include those with a site of action is within CNS.

Examples of drugs with CNS action with or without rapid onset include acids, bases or insoluble salts of drugs such as aprepitant, anti-stroke agents such as clopidogrel (and insoluble bisulphate salt form), nimodipine; antidepressants such as tryptophan, mianserin, moclobemide, isocarboxazid, phenelzine, tranylcypromine, duloxetine, mirtazepine, amitriptyline, clomipramine, dothiepin, imipramine, lofepramine, maprotiline, nortriptyline, protriptyline, trimipramine, doxepin, citalopram, escitalopram, fluoxetine, fluvoxamine, paroxetine, reboxetine, venlafaxine, sertraline, nefazodone, trazodone, hypericum perforatum; anti-cholinergics and anti-muscarinics such as benzhexol, trihexyphenidyl, benztropine, orphenadrine, procyclidine; benzodiazepine anxiolytics/hypnotics such as alprazolam, bromazepam, chlordiazepoxide, clobazam, desmethylclobazam, clorazepate, diazepam, lorazepam, oxazepam, triazolam, temazepam, nitrazepam, flunitrazepam, flurazepam, loprazolam, lormetazepam; non-benzodiazepine anxiolytics such as buspirone, propranolol, oxprenolol; non-benzodiazepine hypnotics such as chloral, clomethiazole, diphenhydramine, promethazine, zaleplon, zolpidem, zopiclone; anti-psychotics/neuroleptics such as sertindole, sulpride, amisulpride, phenothiazines, such as clozapine, chlorpromazine, fluphenazine, methotrimeprazine, pericyazine, perphenazine, promazine, thioridazine, trifluoperazine, olanzapine, quetiapine, zotepine; thioxanthines such as flupenthixol, zuclopenthixol; butyrophenones such as benperidol, haloperidol, droperidol; other anti-psychotics/neuroleptics such as pimozide, aripiprazole; dehydroaripiprazole; anti-cholinesterases such as donezepil, galantamine, rivastigmine; anti-epileptics such as carbamazepine, oxcarbazepine, valproic (acid); phenytoin, gabapentin, pregabalin, tiagabine, vigabatrin, phenobarbital, primidone, lamotrigine; ADHD drugs such as methylphenidate, amphetamines such as dexamphetamine; analgesics such as morphine, codeine and other opioids or opiate derivatives such as oxycodone, oxymorphone, hydrocodone, hydromorphone, diamorphine, dihydrocodeine, dipipanone, ethylmorphine, buprenorphine, methadone, levomethadone, naloxone, naltrexone, nalbuphine, nicomorphine, pentamorphone, pethidine, fentanyl, alfentanil, carfentanil, remifentanil, sufentanil, trefentanil; aceclofenac, ampiroxicam, aspirin (acid), benorylate, benoxaprofen, bezitramide, bromfenac, bufexamac, bumadizone, bupivocaine, levobupvacaine, lidocaine, prilocalne, procaine, tetracaine, ropivacaine; smoking cessation such as nicotine, bupropion, butibufen, butorphanol, capsaicin, carbaspirin, carprofen, dextromoramide, dextropropoxyphene, diclofenac, diflunisal, droxicam, etodolac, etorphine, felbinac, fenbufen, fenclofenac, fenoprofen, flunoxaprofen, flurbiprofen, furprofen, ibufenac, ibuprophen, ibuproxam, imidazole, indomethacin, indoprofen, isoxicam, ketoprofen, ketorolac, ropinirole, lonazolac, lornoxicam, loxoprofen, lysine aspirin, meclofenamate, mefanamic (acid), meloxicam, mofezolac, nabumetone, naproxen (acid), nefopam, nicoboxil, nifenazone, oxindanac, oxyphenbutazone, paracetamol, pentazocine, phenazocine, phenazone, phenylbutazone, piketaprofen, pirazolac, piritramide, piroxicam, pirprofen, pranoprofen, propacetamol, sulindac, suprofen, tenoxicam, tramadol, zaltoprofen, zomepirac.

Examples of non-CNS drugs having systemic action include acids, bases or insoluble salts of drugs of sildenafil, tadalafil, vardenafil, isosorbide, dicycloverine, hyoscine, alverine, loperamide, amiloride, amiodarone, propranolol, bisoprolol, carvedilol, celeprolol, esmolol, labetalol, metoprolol, oxprenolol, sotalol, pindolol, nadolol, atenolol, timolol, hydralazine, candesartan, losartan, olmesartan, amlodipine, diltiazem, dopamine, dopexamine, warfarin (acid) colestipol, salbutamol, terbutaline, bambuterol, fenoterol, formoterol, salmeterol, ephedrine, orciprenaline, ipratropium, tiotropium, glycopyrronium, beclomethasone, fluticasone, mometasone, desloratadine, fexofenadine, loratadine, alimemazine, bromphiramine, chlorpheniramine, cyproheptadine, diphenhydramine, hydroxyzine, promethazine, triprolidine, doxapram, mecysteine, pseudoephedrine, almotriptan, naratriptan, rizatriptan, sumatriptan, zolmitriptan, isometheptene, clonidine, lamotrigine, tiagabine, benzatropine, orphenadrine, procyclidine, memantine, abacavir, didanosine, tenofovir, amantadine, oseltamivir, dexamethasone, betamethasone, cortisone, hydrocortisone, methylprednisolone, prednisolone, prednisone, triamcinolone, medroxyprogesterone, testosterone, cyproterone, alfuzosin, prazosin, tamsulosin, bethanechol, distigmine, flavoxate, oxybutynin, propantheline, propiverine, tolterodine, trospiran, levobupivacaine, bupivacaine, prilocalne, procaine, tetracaine, ropivacaine and lidocaine.

Examples of non-CNS drugs having rapid systemic action include acids, bases or insoluble salts of drugs of sildenafil, tadalafil, vardenafil, levobupivacaine, bupivacaine, prilocalne, procaine, tetracaine, ropivacaine, lidocaine, iloprost, clonidine, guanethidine, alteplase, clopidogrel, hyoscine, alverine, loperamide, salbutamol, terbutaline, bambuterol, fenoterol, formoterol, salmeterol, desloratadine, fexofenadine, loratadine, alimemazine, bromphiramine, chlorpheniramine, pseudoephedrine, almotriptan, naratriptan, rizatriptan, sumatriptan, zolmitriptan, isometheptene, clonidine, lamotrigine, tiagabine, famotidine, rabeprazole, pantoprazole, cimetidine, ranitidine, lansoprazole, esomeprazole, omeprazole.

7) Acid/GI labile drugs.

Examples of such drugs include proteins and peptides (e.g. insulin, calcitonin, heparin, etc.) and drugs conventionally presented in or benefiting from enteric coating.

8) Drugs taken into body via lipid uptake mechanism.

Examples of such drugs include cyclosporine and glatiramer.

9) Drugs, particularly when in submicron form, whether in poorly soluble base form, acid form or a particular salt form.
10) Drugs, particularly when delivered in combination with one or more of surfactants, oils, alcohols, whether in poorly soluble base form, acid form or a particular salt form.
11) Drugs that, particularly when in submicron form, whether in poorly soluble base form, acid form or a particular salt form, fall into the FDA (CDER) ‘biopharmaceutical classification system (BCS)’ category: Class II—High Permeability, Low Solubility.

Examples of such Class II drugs include glibenclamide, phenytoin, danazol, ketoconazole, mefenamic acid, nifedipine, rifampicin, ethambutol, pyrazinamide, isoniazid, quinidine, chloroquine, mebendazole, niclosamide, prasiquantel, atenolol, piroxicam and amitriptyline.

12) Drugs that, particularly when delivered in combination with one or more of surfactants, oils, alcohols, whether in poorly soluble base form, acid form or a particular salt form, fall into the FDA (CDER) ‘biopharmaceutical classification system (BCS)’ category: Class II—High Permeability, Low Solubility.

Examples of such Class II drugs are given above.

13) Drugs that, particularly when delivered transmucosally, especially via sublingual/buccal mucosa, fall into the FDA (CDER) ‘biopharmaceutical classification system (BCS)’ category: Class III—Low Permeability, High Solubility.

Examples of such Class III drugs include proteins and peptides, cimetidine, ranitidine, acyclovir, neomycin B, captopril, ketoprofen, naproxen (acid form), carbamazepine, ciprofloxacin, valsartan (acid form), as well as olmesartan, candesartan, bosentan, telmisartan, losartan, irbesartan, etc. (all in acid form).

14) Drugs, particularly those in base form and delivered transmucosally, especially via sublingual/buccal mucosa, that fall into the FDA (CDER) ‘biopharmaceutical classification system (BCS)’ category: Class IV—Low Permeability, Low Solubility.

Examples of such Class IV drugs include taxol, hydrochlorothiazide and furosemide.

15) Drugs presented in submicron form and delivered transmucosally that fall into the FDA (CDER) ‘biopharmaceutical classification system (BCS)’ category: Class III—Low Permeability, High Solubility.

Examples of such Class III drugs are given above.

16) Drugs that, when delivered transmucosally in combination with one or more of surfactants, oils, alcohols, fall into the FDA (CDER) ‘biopharmaceutical classification system (BCS)’ category: Class III—Low Permeability, High Solubility.

Examples of such Class III drugs are given above.

17) Drugs presented in submicron form and delivered transmucosally that fall into the FDA (CDER) ‘biopharmaceutical classification system (BCS)’ category: Class IV—Low Permeability, Low Solubility.

Examples of such Class IV drugs are given above.

18) Drugs that, when delivered transmucosally in combination with one or more of surfactants, oils, alcohols, fall into the FDA (CDER) ‘biopharmaceutical classification system (BCS)’ category: Class IV—Low Permeability, Low Solubility.

Examples of such Class IV drugs are given above.

19) Drug molecules for non-central systemic delivery with a polar surface area greater than 60 Å² presented in submicron base form for transmucosal delivery.
20) Drug molecules for delivery via systemic circulation to CNS with a polar surface area less than 140 Å², presented in submicron base form for transmucosal delivery.
21) Drugs that require uptake into the systemic circulation via an active transporter mechanism and where modification or blockade of this transporter mechanism by other drugs or by high concentrations of the same drug adversely affects absorption e.g. gabapentin, pregabalin and baclofen.

It should be noted that transmucosal delivery, especially in the head and neck region, may assist the administered drug reaching a site of action within the CNS, because the blood flow in this region can allow active agents to reach the cranial arteries promptly in higher concentrations and without first passing either the liver or other body volumes.

The following therapeutic classes of drugs are examples of drug types and specific drugs that have qualities that make them particularly suitable for incorporation into the compositions according to the present invention. All of the drugs mentioned are already registered in the more soluble salt form. Except where specified, the drugs listed below all refer to a possible base form that could be used more beneficially than salt forms in the present invention, for the reasons set out above.

1. Drugs for treating acid-peptic and motility disorders, laxatives, antidiarrhoeals, colorectal agents, pancreatic enzymes and bile acids.
2. Drugs for treating arrhythmias and cardiac failure, anti-anginals, diuretics, antihypertensives, drugs for treating circulatory disorders, anticoagulants, antithrombotics and fibrinolytics, haemostatics, hypolipidaemic agents, drugs for treating anaemia and neutropenia.
3. Hypnotics, anxiolytics, antipsychotics, antidepressants and mood stabilisers, antiemetics, anticonvulsants, drugs for treating neurodegenerative diseases, drugs for modulating sleep architecture, and drugs for treating ADHD and narcolepsy.
4. Analgesics, anti-pyretics and migraine treatments.
5. Drugs for treating musculo-skeletal disorders, NSAIDs, disease modifying antirheumatic drugs, drugs for treating gout, muscle relaxants, neuromuscular drugs.
6. Drugs for treating male sexual disorders, corticosteroids, growth hormones, drugs for treating growth disorders, thyroid and antithyroid drugs, drugs affecting bone metabolism, drugs for treating diabetes insipidus.
7. Insulin, oral hypoglycemics, drugs for treating hypoglycaemia.
8. Drugs for treating infections and infestations, antibiotics and antibacterials, antifungals, antituberculosis and antileprotics, antimalarials, anthelmintics and amoebicides, drugs for treating herpes, drugs for treating hepatitis and other viral infections, vaccines and immunoglobulins, immunomodulators.
9. Drugs for treating genital infections, urinary tract infections, renal and bladder infections.
10. Drugs for treating inborn errors of metabolism, anti-obesity agents.
11. Bronchodilators and anti-inflammatory drugs, expectorants, antitussives, mucolytics and decongestants.
12. Local reactants on nose, oropharyngeal preparations, aural preparations.
13. Ocular anti-infectives and anti-inflammatories, drugs for treating glaucoma, ocular lubricants.
14. Anti-allergic drugs, hyposensitising preparations.
15. Contraceptive drugs.
16. Drugs for treating cancer.
17. Drugs for treating dysmenorrhoea, menorrhagia, endometriosis, premenstrual disorders, breast disorders, menopausal disorders, obstetrics, infertility.
18. Drugs for treating poisoning, drug and alcohol dependency.
19. Anaesthetics and premeds.
20. Drugs for treating mucositis.

In an embodiment of the present invention, the composition comprises one or more tri-cyclic antidepressants such as amitriptyline, nortriptyline, clomipramine and imipramine, SSRIs such as fluoxetine, paroxetine, citalopram, escitalopram and sertraline and/or SNRIs such as duloxetine and venlafaxine. Preferably, the composition is for treating depression and/or sleep disorders.

In an embodiment of the present invention, the composition comprises one or more anti-migraine agents such as sumatriptan, frovatriptan, zolmitriptan, eletriptan, almotriptan, dihydroergotamine and/or analgesics such as NSAIDs and paracetamol. Preferably, the composition is for treating or preventing migraine.

In an embodiment of the present invention, the composition comprises one or more of morphine, codeine, other opiates and opioids such as oxycodone, oxymorphone, dihydrocodeine, hydromorphone, hydrocodone, fentanyl, sufentanyl, alfentanyl and buprenorphine, tri-cyclics such as amitriptyline, gabapentin, pregabalin, and analgesics such as NSAIDs and paracetamol. Preferably, the composition is for treating or preventing pain.

In an embodiment of the present invention, the composition comprises one or more anxiolytics and/or hypnotics such as benzodiazepines such as desmethylclobazam and non-benzodiazepines such as buspirone, propranolol, oxprenolol; chloral, clomethiazole, diphenhydramine, promethazine, zaleplon, zolpidem base and zopiclone.

In an embodiment of the present invention, the composition comprises one or more anti-psychotics and/or neuroleptics such as sertindole, sulpride, amisulpride, phenothiazines, benzisoxazoles, thioxanthines, butyrophenones, clozapine, olanzapine, pimozide, aripiprazole, dehydroaripiprazole, and anti-cholinesterases.

In an embodiment of the present invention, the composition comprises one or more anti-convulsants including benzodiazepines, carbamazepine, oxcarbazepine, valproic (acid); phenytoin, gabapentin, pregabalin, tiagabine, vigabatrin, phenobarbital, primidone and lamotrigine.

In an embodiment of the present invention, the composition comprises one or more anti-emetics including 5HT3 antagonists such as palonosetron, dolasetron, ondansetron, granisetron, tropisetron, anticholinergics such as hyoscine, anti-dopaminergics such as metoclopramide, prochlorperazine, promethazine and NK-1 antagonists such as aprepitant. Preferably, the composition is for treating or preventing emesis.

In an embodiment of the present invention, the composition comprises one or more drugs including acamprosate, taurine, naltrexone, methadone, buprenorphine, naloxone, nicotine, bupropion, cytisine and varenicline base. Preferably, the composition is for treating drug dependency.

In an embodiment of the present invention, the composition comprises one or more drugs including PDE5 inhibitors such as sildenafil base, tadalafil and vardenafil, dopamine agonists such as apomorphine, alprostadil, SSRIs such as fluoxetine, paroxetine, citalopram, escitalopram and sertraline), SNRIs such as duloxetine and venlafaxine, TCAs such as nortriptyline, clomipramine and lofepramine and trazodone. Preferably, the composition is for treating sexual dysfunction.

In an embodiment of the present invention, the composition comprises one or more drugs including anti-platelet agents such as tirofiban, eptifibatide, abciximab, clopidogrel and dipyridamole, anti-coagulants such as heparins, heparinoids and prostaglandins, angiotensin II agonists such as irbesartan, candesartan, losartan and olmesartan. Preferably, the composition is for treating conditions associated with CVA, angina or myocardial infarction.

In an embodiment of the present invention, the composition comprises one or more drugs including ACE inhibitors, beta blockers, nifedipine, nimodipine, prazosin, nicotinic acid, inositol nicotinate, moxisylyte, cilostazol, xanthine and naftidrofuryl base. Preferably, the composition is for treating circulatory disorders, such as Raynauds disease.

In an embodiment of the present invention, the composition comprises one or more oral hypoglycaemic drugs including thiazolidinediones such as pioglitazone and rosiglitazone, biguanides such as metformin, sulphonylureas such as glipizide, nateglinide, repaglinide and insulin.

### EXAMPLE 1A

### Sumatriptan Formulation Preparation (Spray Drying)

This example relates to spray dried sumatriptan formulation. The target batch size was a minimum of 200 g of spray dried powder. To produce in excess of 200 g of spray dried powder, it was envisaged that up to 400 g of solids would have to be spray dried (based on 50% recovery). At a feed concentration of 12.5 g/L this equated to a liquid fee volume of 32 L (spray drying feed at 1.25% (w/v) solids) and an estimated spray drying time of 22 hours.

Because of the relatively long spray drying times (and the need to clear the filter bag after 6-8 hours), a series of batches were spray dried and then pooled at the end of processing with the minimum target recovery of 200 g. The first two batches were to have 150 g sumatriptan in a feed volume of 12 L, providing an expected yield of 75 g (based upon a 50% recovery). The amount spray dried in the third batch was then adjusted dependant on recoveries from the first 2 batches.

The following materials were used:

| Chemical/Grade | Supplier | Manufacturer's Batch No. |
|---|---|---|
| Sumatriptan USP | SMS Pharmaceutical Ltd. | SMT/07 06 003 |
| Tween 80 | Croda | Not available |
| Maltitol | Rocquette | 4792555 |
| Polydextrose | Danisco | 129966-IP-V63030P |
| Lutrol F127 | BASF | Koll 3003093566 |
| HPMC/5 | Colorcon | Lot SC23012403 |
| Ethanol ABS | Liverpool University | Not available |
| DI Water | Upperton Ltd | Not available |

Spray drying was carried out using a Niro Mobile Minor modified for pharmaceutical applications. The drying air fan was fitted upstream to the spray dryer, i.e. the dryer was run under positive drying chamber pressure. The lid was sealed with pressure-resistant clamps. Atomisation was achieved using a Niro 2-fluid air atomisation nozzle (air pressure provided by a Hydrovane oil-free compressor). The liquid feed was achieved using an IsmaTec® gear pump capable of up to 100 ml/min feed rate.

Preparing the batches, the following chemicals were weighed out and stored in a separate sealed container:

| | Amount (g) | | |
|---|---|---|---|
| Chemical/Grade | Batch 25#37/01 | Batch 25#37/02 | Batch 25#37/03 |
| Sumatriptan USP | 60.02 | 60.06 | 14.99 |
| Tween 80 | 9.04 | 9.03 | 2.28 |
| Maltitol | 9.01 | 9.00 | 2.25 |
| Polydextrose | 9.02 | 9.03 | 2.25 |
| Lutrol F127 | 9.00 | 9.02 | 2.26 |
| HPMC/5 | 54.04 | 54.03 | 13.49 |

Sumatriptan was added to 7.2 L ethanol (1.8 L for Batch 25#37/03) and left to stir overnight at room temperature.

On the next day, the HMPC was added to the sumatriptan solution and stirred to produce an even suspension. An aqueous solution was then prepared by adding the following solutes to 4.8 L (1.2 L for Batch 25#37/03) de-ionised water:maltitol, polydextrose, Lutrol F127, Tween 80 in the amounts given in the table above. The aqueous solution was added to the sumatriptan/HPMC suspension and the resulting suspension turned to a clear, yellow solution.

The Niro Mobile Minor was set up for use and equilibrated (using 50% ethanol solution as a liquid feed) with the following settings:

| | Batch No. | | |
|---|---|---|---|
| | 25#37/01 | 25#37/02 | 25#37/03 |
| Inlet temperature (start) | 100° C. | 100° C. | 100° C. |
| Outlet temperature (start) | 61° C. | 60° C. | 60° C. |
| Liquid feed rate | 25 ml/min | 25 ml/min | 25 ml/min |
| Atomisation pressure (start) | 0.7 bar | 0.75 bar | 0.75 bar |
| Atomisation air flow (start) | 74 L/min | 80 L/min | 74 L/min |
| Drying chamber pressure (start) | 85 mmWS | 85 mmWS | 85 mmWS |

The sumatriptan solution was spray dried at these settings. For Batches 25#37/01 and 02 the product collection jar was replaced and the contents recovered on 4 occasions. After all of the solution had been atomised, drying was halted and the spray dried powder recovered. At the end of the spray drying run, the following parameters were recorded:

| | Batch No. | | |
|---|---|---|---|
| | 25#37/01 | 25#37/02 | 25#37/03 |
| Inlet temperature (end) | 100° C. | 100° C. | 100° C. |
| Outlet temperature (end) | 61° C. | 61° C. | 64° C. |
| Liquid feed rate (end) | 25 ml/min | 25 ml/min | 25 ml/min |
| Atomisation pressure (end) | 0.7 bar | 0.75 bar | 0.75 bar |
| Atomisation air flow (end) | 80 L/min | 80 L/min | 80 L/min |
| Drying chamber pressure (end) | 95 mmWS | 95 mmWS | 95 mmWS |

The spray dryer was cleaned and dried prior to further use. For each sample of spray dried material produced, 25 mg of the sumatriptan-containing powder was dispersed into 26 ml distilled water. Occasionally, a vortex mixer was used to help dispersion. The particle size in solution was measured using a Malvern Nano S Instrument. Particle sizing measurements were performed in triplicate which allows sizes to be averaged and a standard deviation to be calculated. Measurements were only judged to be accurate if the standard deviation between three results was less than 10%.

For each sample, 50 mg of powder (containing 20 mg, equivalent to one dose) was dissolved into 1000 ml of distilled water at 37° C. with overhead paddle stirring at 50 rpm. Aliquots of each solution were taken at 5 min, 10 min and 15 min. These dispersions were then diluted with 0.1 mol/1 HCl solution for UV characterisation. From the data obtained the percentage dissolution was calculated.

The recoveries obtained from the two runs are shown below:

| | Quantity of Material Recovered |
|---|---|
| Batch No. | (% of starting material) |
| 25#37/01 | 108.9 g (73%) |
| 25#37/02 | 110 g (73%) |
| 25#37/03 | 25.3 g (68%) |

The powder particle sizes were generated using a Sympatec Helos Laser Sizer that calculates the particle size based upon laser diffraction. The sizing is done on dry powder samples dispersed in an air stream.

The particles were dispersed as a dry powder in a stream of compressed air (known as the Rodos dry powder disperser). Approximately 50 mg of the powder was fed into the Rodos using an Aspiros deliver unit.

The samples were sized using an air dispersal pressure of 5 bar, it having been established that pressures between 3 and 6 bar were enough to completely disperse the powder without damaging primary particle size. The laser diffraction pattern was collected with a lens that had a range between 0.5 and 175 μm.

| Sample No. | Particle Size |
|---|---|
| Sample 25#37/01 | 12.85 μm |
| Sample 25#37/02 | 12.41 μm |
| Sample 25#37/03 | 12.63 μm |

The samples were then characterised in terms of dissolution speed and particle size when dispersed in water. Initially the dissolution speed of the samples was measured as received. The results were as follows:

| | Dissolution | Dissolution | Dissolution | |
|---|---|---|---|---|
| | at 5 mins | at 10 mins | at 15 mins | Equilibrium |
| Batch No. | (wt %) | (wt %) | (wt %) | wt % |
| 05/25/54-UT | 100 | 99.9 | 99.5 | 100 |
| 04 | | | | |
| 05/25/54-UT | 100 | 100 | 100 | 100 |
| 05 | | | | |
| 05/25/54-UT | 92 | 98 | 99 | 100 |
| 06 | | | | |

The data obtained from all three samples was quite consistent, although the last batch was slower to dissolve during the first 5 minutes compared to the other batches. In addition, the last batch also contained some larger powder particles. More than 90 wt % of each of the powders was recovered after sieving. The sieved powders were then dried at room temperature under vacuum for 12 hours to ensure no residual solvents remained (to aid stability of the powder). Dissolution data was then recorded for the dry, sieved powders.

| | Dissolution | | | |
|---|---|---|---|---|
| | at 5 mins | Dissolution at | Dissolution at | Equilibrium |
| Batch No. | (wt %) | 10 mins (wt %) | 15 mins (wt %) | wt % |
| 05/25/54- | 100 | 100 | 100 | 100 |
| UT 04 | | | | |
| 05/25/54- | 98.1 | 99.2 | 100 | 100 |
| UT 05 | | | | |
| 05/25/54- | 99.5 | 100 | 100 | 100 |
| UT 06 | | | | |

The dissolution data for the dry and sieved samples was much more consistent with almost complete dissolution within 5 minutes.

The moisture content (water and/or any residual solvent) of the samples was calculated by measuring the loss of weight upon drying the samples under vacuum, at room temperature in a standard laboratory vacuum oven for 12 hours. After this drying step, it was assumed that the volatile portion of the material had been removed and that the material was dry. As shown by the results below, the samples had less than 1 wt % moisture/residual solvent.

| Batch No. | Weight Loss |
|---|---|
| 05/25/54-UT 04 | 0.5 wt % |
| 05/25/54-UT 05 | 0.5 wt % |
| 05/25/54-UT 06 | 0.4 wt % |

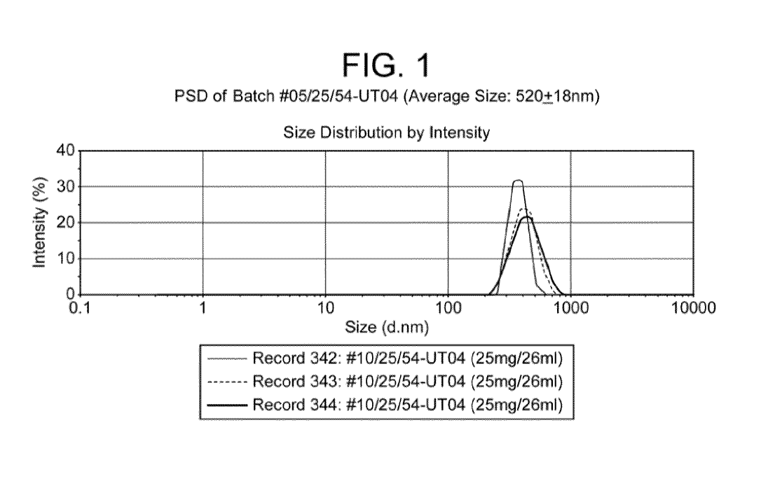

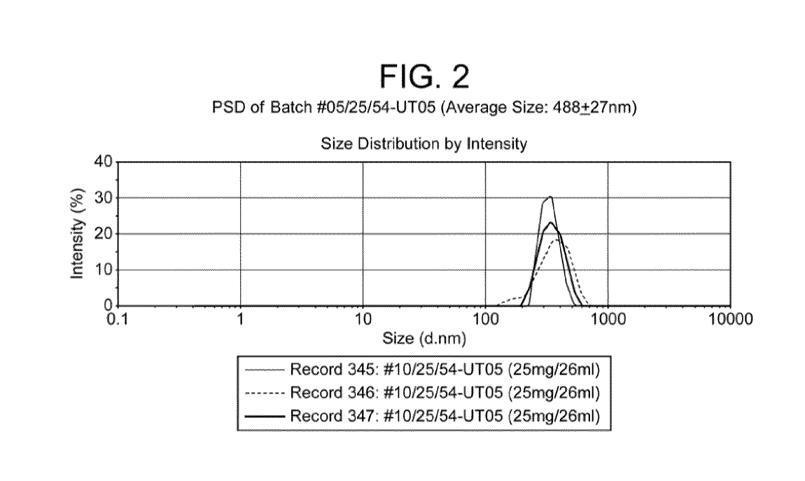

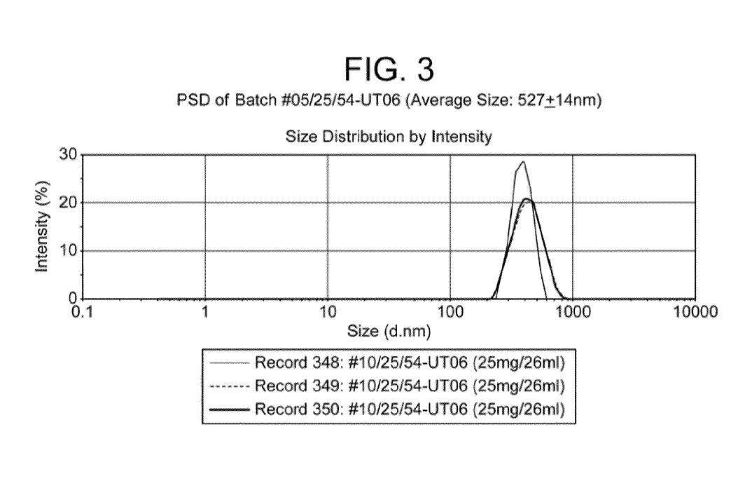

The particle size was recorded for each batch (after drying and sieving) at a concentration of 25 mg/26 ml of water. The data is shown in FIGS. 1, 2 and 3. FIG. 1 shows the particle size distribution of Batch 05/25/54-UT 04, which had an average particle size of 520±18 nm. FIG. 2 shows the particle size distribution of Batch 05/25/54-UT 05, which had an average particle size of 488±27 nm. FIG. 3 shows the particle size distribution of Batch 05/25/54-UT 06, which had an average particle size of 527±14 nm. Again, the three batches are very reproducible, although it was noted that the second batch (05/25/54-UT 05) gave slightly smaller particle size than did the other two batches.

Following drying, submicron particles were dry blended with other inert ingredients to produce an organoleptically acceptable powder for administration.

### EXAMPLE 1B

### Sumatriptan Formulation Preparation (Spray Drying)

This example relates to further spray dried sumatriptan formulations.

The following materials were used:

| | | Pharmacopoeia |
|---|---|---|
| Chemical/Grade | Supplier | Conformity |
| Sumatriptan Base | S&D Chemical, India | USP (Sumatriptan |
| | | Base) |
| Methocel E5 Premium | Colorcon, UK | EP (Hypromellose) |
| (HPMC 5) | | USP (Hypromellose |
| | | 2910) |
| MaltitolLitesse ® II IP | Danisco Deutschland, | FCC grade |
| Powder (Polydextrose) | Germany | |
| Maltisorb P 90 | Roquette Frères, France | EP (Maltitol) |
| (Malititol) | | |
| Lutrol F 127 | BASF ChemTrade, | EP (Poloxamer) |
| | Germany | USP (Poloxamer) |
| Tween ® 80 | VWR International, | EP (Polysorbate 80) |
| | Switzerland | USP (Polysorbate 80) |
| Ethanol Anhydrous | Alcosuisse, Switzerland | EP (Ethanol |
| | | Anhydrous) |
| Purified Water USP | Micro-Sphere, | USP (Purified Water) |
| | Switzerland | |

| Chemical | Amount (g) |
|---|---|
| Sumatriptan Base | 40 |
| Ethanol | 3800 (3.8 kg) |
| HPMC 5 | 36 |
| Tween 80 | 6 |
| Maltitol | 6 |
| Polydextrose | 6 |
| Lutrol F127 | 6 |
| Purified Water | 3200 (3.2 kg) |

Sumatriptan was added to the ethanol anhydrous and left to stir overnight at room temperature (19-25° C.).

On the next day, the HMPC 5 was added to the sumatriptan solution and stirred for one hour to produce an even suspension. An aqueous solution was then prepared by adding the maltitol, polydextrose, Lutrol F127, Tween 80 to the purified water and stirring for one hour. The aqueous solution was added to the sumatriptan/HPMC suspension and was stirred for 30 minutes, resulting in a clear solution. The solution was then spray dried using a Niro Mobile Minor™ 2000 spray drying plant equipped with a cyclone and a cartridge filter. The drying gas, compressed air, is heated by an electrical heater and enters the drying chamber through a ceiling air disperser. A peristaltic pump with silicone hoses pumps the feed to a two-fluid nozzle, placed in the top of the chamber. The resultant product is discharged from the bottom of the cyclone in an antistatic polyethylene bag.

The spray drying was conducted under the following parameters:

| | Batch No. 1 | Batch No. 2 | Batch No. 3 | Batch No. 4 | Batch No. 5 |
|---|---|---|---|---|---|
| Inlet temperature | 100° C. | 102° C. | 102° C. | 101° C. | 101° C. |
| Outlet temperature | 58° C. | 58° C. | 57° C. | 57° C. | 57° C. |
| Liquid feed rate | 15 g/min | 17 g/min | 17 g/min | 18 g/min | 18 g/min |
| Atomisation pressure | 0.7 bar | 0.7 bar | 0.7 bar | 0.7 bar | 0.7 bar |
| Drying air flow | 2.5 mbar | 2.6 mbar | 2.5 mbar | 2.5 mbar | 2.5 mbar |
| | (75 kg/h) | (75 kg/h) | (75 kg/h) | (75 kg/h) | (75 kg/h) |
| Atomisation air flow | 49% (3.9 kg/h) | 56% (4.5 kg/h) | 53% (4.2 kg/h) | 45% (3.6 kg/h) | 55% (4.4 kg/h) |

### Batch 1:

After approximately 1 hour of spray drying the antistatic polyethylene bag was changed, in order to have a sample (Bag 1) for in process analytical testing. The same process with adopted after approximately 3 hours of spray drying, because of a tiny hole in the collection bag. The antistatic bag with the hole (Bag 2) was replaced with a new bag (Bag 3).

The contents of the three bags were mixed together, put on a stainless steel tray and dried in the vacuum drying oven overnight at room temperature and 200 mbar absolute pressure.

The equipment used for vacuum drying was a Kendro VT 6130 M vacuum drying oven equipped with stainless steel trays and a Vacuubrand MZ 2C vacuum pump. A slight flow of nitrogen was left to enter the oven throughout the vacuum drying phase. The next day, the vacuum dried powder was weighed and packed in two antistatic polyethylene bags.

The product recovered in the antistatic bag was then put on a stainless steel plate and dried in a vacuum oven overnight at room temperature and 200 mbar absolute pressure. A slight flow of nitrogen was allowed to enter the oven throughout the vacuum drying phase. Following the drying step, the vacuum dried powder was weighed and packed into two antistatic bags.

### Batches 2 and 3:

The product recovered in the antistatic polyethylene bag was put on a stainless steel plate and dried in the vacuum drying oven overnight at room temperature and 200 mbar absolute pressure. A slight flow of nitrogen was left to enter the oven throughout the vacuum drying phase. The next day, the vacuum dried powder was weighed and packed in to antistatic polyethylene bags.

### Batches 4 and 5:

The spray dried product collected in the antistatic polyethylene bag attached to the cyclone was weighed and packed in two antistatic polyethylene bags.

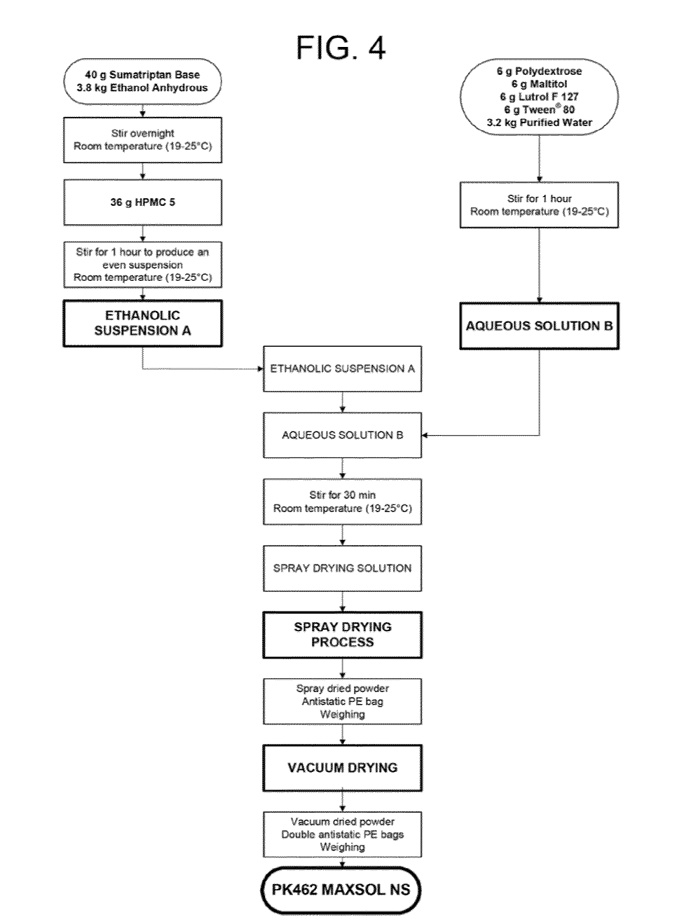

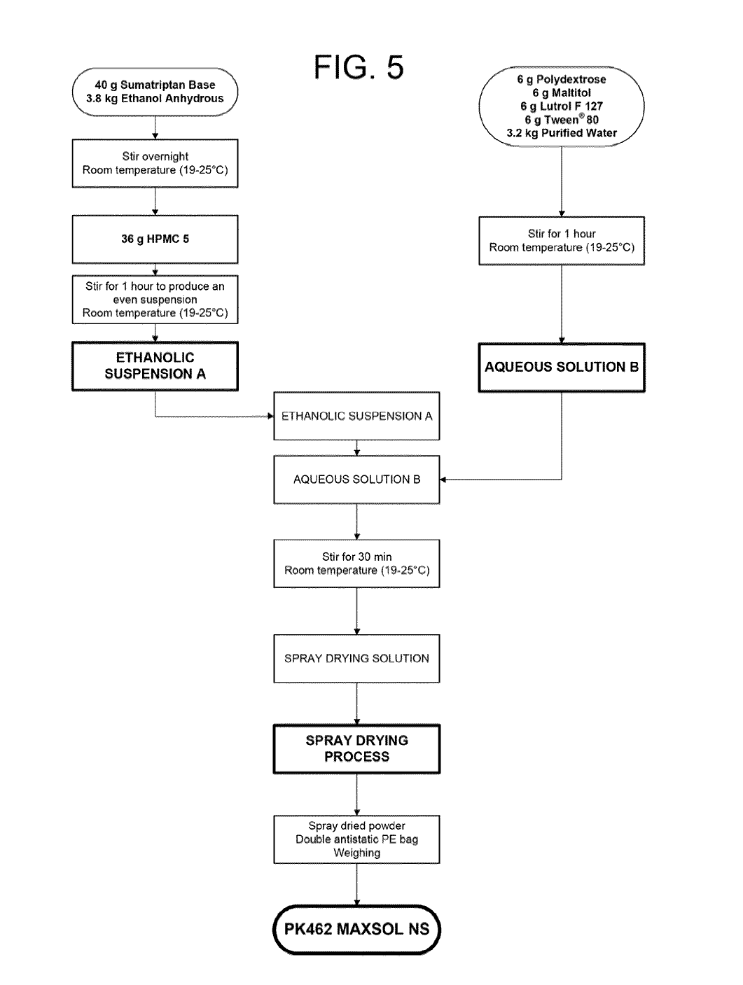

The process used to prepare Batches 1, 2 and 3 is summarised in the flowchart shown in FIG. 4, whilst the process used to prepare Batches 4 and 5 is summarised in the flowchart shown in FIG. 5,

### Results

### Recovery of Spray Dried Powders

Quantity of material recovered
(% of starting material)

| | | |
|---|---|---|
| Batch 1 | After spray drying: | Bag 1: 6.5 g |
| | | Bag 2: 18 g |
| | | Bag 3: 34 g |
| | Total: | 58.5 g (58.5%) |
| | After vacuum drying: (final) | 48 g (48%) |
| Batch 2 | After spray drying: | 66.5 g (66.5%) |
| | After vacuum drying: (final) | 61 g (61%) |
| Batch 3 | After spray drying: | 62 g (62%) |
| | After vacuum drying: (final) | 59.5 g (59.5%) |
| Batch 4 | After spray drying: | 62 g (62%) |
| | No vacuum drying | |
| Batch 5 | After spray drying: | 62 g (62%) |
| | No vacuum drying | |

The recovery of the product after spray drying was not significantly different between the batches. There was some product loss after vacuum drying (see Batches 1-3).

Furthermore, the decrease in moisture content (discussed in the next paragraph) is not high enough to justify such high product losses.

### Moisture Content (Loss on Drying)

Between 1 and 2 g of the spray dried powder was put onto the Mettler-Toledo LJ 16 thermobalance, code MS-301. The powder was dried for 20 minutes at 70° C.

| | Sample | Loss on drying (%) |
|---|---|---|
| Batch 1 | Bag 1 | 1.81% |
| | Final (vacuum dried) | 1.58% |
| Batch 2 | Spray Dried | 1.79% |
| | Final (vacuum dried) | 1.43% |
| Batch 3 | Spray Dried | 1.20% |
| | Final (vacuum dried) | 0.80% |
| Batch 4 | Final (spray dried) | 1.37% |
| Batch 5 | Final (spray dried) | 0.74% |

The loss on drying decrease after overnight drying in the vacuum drying oven is around 0.23-0.4%, i.e. from 13% to 33% with respect to the loss on drying value after spray drying.

### Particle Size Measurement Spray Dried Powder Particle Size

Size analysis of the spray dried batches were carried out using a Sympatec Helos laser sizer, fitted with a Rodos air dispenser, code App. 106B. A few hundred milligrams of powder was fed into the disperser using a vibratory feeder and dispersed at 5.0 bar dispersal pressure.

| | Sample | X₁₀ | X₅₀ | X₉₀ |
|---|---|---|---|---|
| Batch 1 | Bag 1 | ≦2.63 μm | ≦8.99 μm | ≦34.3 μm |
| | Bag 2 | ≦1.87 μm | ≦6.31 μm | ≦18.23 μm |
| | Final (vacuum dried) | ≦1.86 μm | ≦6.97 μm | ≦21.18 μm |
| Batch 2 | Spray Dried | ≦2.37 μm | ≦7.62 μm | ≦19.08 μm |
| | Final (vacuum dried) | ≦2.30 μm | ≦7.22 μm | ≦17.54 μm |
| Batch 3 | Spray Dried | ≦2.82 μm | ≦8.26 μm | ≦22.44 μm |
| | Final (vacuum dried) | ≦2.59 μm | ≦7.22 μm | ≦19.97 μm |
| Batch 4 | Final (spray dried) | ≦1.80 μm | ≦10.10 μm | ≦35.33 μm |
| Batch 5 | Final (spray dried) | ≦2.08 μm | ≦7.20 μm | ≦16.96 μm |

During the preparation of Batch 1, the feed rate was increased from 14 g/min to 17 g/min after collecting the Bag 1 sample. This is probably the reason why the size of the Batch 1 particles decreased between Bag 1 and the final (vacuum dried) sample.

### Particles Size Measurement Nano-Particle Sizing

25 mg (balance Sartorius A 200S, code MS-209) of each powder was dispersed into 20 ml demineralised water using a vortex mixer (Vortes Genie 2 G560E, code MS-181) to assist dispersion. 3 ml of the dispersion was placed into a cuvette and sonicated for 3 seconds (Bandelin Sonorex RK 156, code MS-328) to remove any air bubbles. The cuvetter was then introduced into the Sympatec NANOPHOX, code App. 111 for nanoparticule analysis at 21.5° C. Particle sizing measurements were performed in triplicate unless otherwise stated.

| | Sample | Average Size | |
|---|---|---|---|
| Batch 1 | Bag 1 | 587 | nm (one analysis only) |
| | Bag 2 | 697 | nm (one analysis only) |
| | Final (vacuum dried) | 590 ± 5 | nm |
| Batch 2 | Final (vacuum dried) | 598 ± 5 | nm |
| Batch 3 | Final (vacuum dried) | 620 ± 15 | nm |
| Batch 4 | Final (spray dried) | 682 ± 7 | nm |
| Batch 5 | Final (spray dried) | 622 ± 5 | nm |

There was no significant difference between the batches.

UV Assay

For each sample, 12.5 mg of powder (containing 5 mg of sumatriptan) was weighed using a Sartorius A 200 S balance, code MS-209, and transferred to a 100-ml volumetric flask. 100 ml of 0.1 mol/1 HCl solution was then places into the 100-ml flask and mixed until complete dissolution. Aliquots of sumatriptan solution were introduced into the Cecil CE 3021 UV-Vis Spectrophotometer, code App. 104B. A range of concentrations of sumatriptan were prepared in 0.1 mol/1 HCl solution in order to acquire a calibration curve. The range of concentrations of sumatriptan used for the UV calibration curve was from 0.0016 mg/ml to 0.08 mg/ml. The curve was linear through the whole concentration range. The sumatriptan solutions were quantified by UV at 283 nm. UV assay measurements were performed in triplicate.

| | US Assay at 283 nm (% of declared) |
|---|---|
| Batch 1 | 397.46 ± 2.27 mg/g (99.37%) |
| Batch 2 | 395.21 ± 1.48 mg/g (98.80%) |
| Batch 3 | 386.74 ± 2.56 mg/g (96.69%) |
| Batch 4 | 388.09 ± 1.74 mg/g (97.02%) |
| Batch 5 | 386.14 ± 3.25 mg/g (96.54%) |

There was no significant difference between the batches.

### Dissolution Test

For each sample 50 mg of powder (containing 20 mg sumatriptan) was weighed using a Sartorius A 200 S balance, code MS-209. the water bath of the Sotax AT 7 Smart dissolution test, code MS-334 was set at 37° C.1000 ml of demineralised water was placed into a dissolution test glass vessel and the stirring was set at 50 rpm, in order to allow the temperature to equilibrate. The temperature equilibriation continued for at least 1 hour. Under continuous stirring, the powder was added into the demineralised water. 2 ml aliquots of each solution were taken by a 2-ml glass pipette at time intervals of 5 minutes, 10 minutes and 15 minutes. The dispersions were then diluted with 0.2 mol/1 HCl solution for UV charaterisation, i.e. 2 ml 0.2 mol/1 HCl was added to 2 ml dispersion (in order to form a 0.1 mol/1 HCl fully molecularly dissolved sumatriptan solution in the acidified aqueous media from which a UV spectra may be obtained).

The sumatriptan solutions were quantified by UV at 283 nm. For the release calculations, the calibration curve obtained for UV assay analysis was used. Dissolution test measurements were performed in duplicate.

| | Dissolution | Dissolution | Dissolution |
|---|---|---|---|
| | at 5 mins | at 10 mins | at 15 mins |
| Batch 1 | 92.92 ± 2.79% | 96.08 ± 0.07% | 97.90 ± 2.64% |
| Batch 2 | 90.63 ± 1.63% | 95.92 ± 0.17% | 99.66 ± 4.28% |
| Batch 3 | 93.55 ± 2.04% | 90.84 ± 1.78% | 92.93 ± 1.34% |
| Batch 4 | 88.08 ± 2.67% | 90.77 ± 1.43% | 93.47 ± 1.88% |
| Batch 5 | 99.13 ± 0.85% | 102.77 ± 0.01% | 101.56 ± 0.86% |

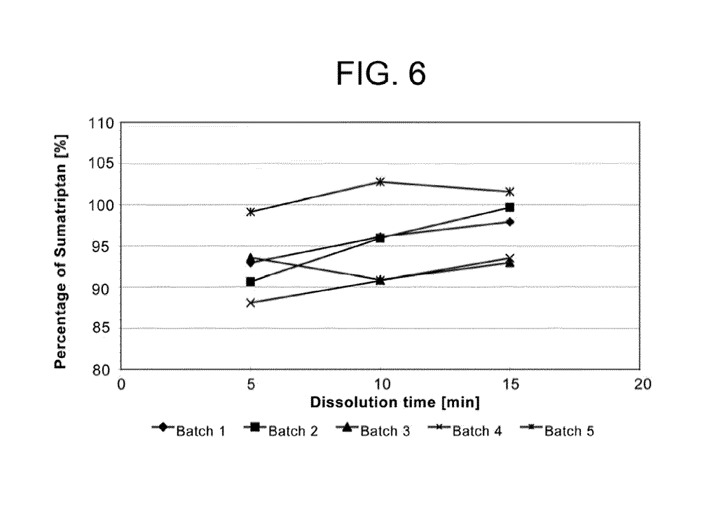

These results are also shown in the graph of FIG. 6. The data obtained from all five batches were quire consistent, although Batch 5 dissolved faster during the first 5 minutes compared to the other batches. In addition, the particles contained in Batch 5 seem to be smaller than those in the other batches, which probably contributed to the faster dissolution rate.

### EXAMPLE 2

### Sumatriptan Formulation Preparation (Co-Milling)

This example relates to co-milled sumatriptan formulation. The target batch size was a minimum of 200 g of co-milled powder. To produce in excess of 200 g of co-milled powder, it was envisaged that approximately 400 g of solids would have to be milled (based on 50% recovery). At a feed rate of 2 g per minute this equated to an estimated co-milling time of approx 3 hours.

Active and inert ingredient components were dry blended using a tumbling blender in order to ensure that the surfactant component was intimately mixed with the sumatriptan powder prior to entry to the mill. The following types and quantities of materials were used:

(a) Co-Milling with Non-Ionic Surfactant

| | Concentration | | |
|---|---|---|---|
| Chemical/Grade | (percent w/w) | | |
| Sumatriptan USP | 95 | 90 | 85 |
| Pluronic | 5 | 10 | 15 |

(b) Co-Milling with Anionic Surfactant

| | |
|---|---|
| | Concentration |
| Chemical/Grade | (percent w/w) |

| | | | | |
|---|---|---|---|---|
| Sumatriptan USP | 99 | 98 | 95 | 90 |
| Sodium lauryl sulphate | 1 | 2 | 5 | 10 |

(c) Co-Milling with Alcohol

| | |
|---|---|
| | Concentration |
| Chemical/Grade | (percent w/w) |

| | | | | |
|---|---|---|---|---|
| Sumatriptan USP | 99.8 | 99 | 98 | 95 |
| Menthol | 0.2 | 1 | 2 | 5 |

(d) Co-Milling with Co-Solvent

| | Concentration | | |
|---|---|---|---|
| Chemical/Grade | (percent w/w) | | |
| Sumatriptan USP | 95 | 90 | 85 |
| Polyethylene Glycol 20,000 | 5 | 10 | 15 |

(d) Co-Milling with Mixed Inert Ingredients

| | Concentration | | |
|---|---|---|---|
| Chemical/Grade | (percent w/w) | | |
| Sumatriptan USP | 94 | 90 | 84 |
| Pluronic | 4 | 9 | 15 |
| Menthol | 2 | 1 | 1 |

Co-milling was carried out using a cryogenic mill (microniser). Following milling, submicron particles were dry blended with other inert ingredients to produce an organoleptically acceptable powder for administration.

### EXAMPLE 3

### Atenolol HCl and Atenolol Base for Peroral Administration—Fast Spreading Mucosal Formulations Preparation

This example relates to freeze dried atenolol HCl and atenolol base formulation. The target batch size was approx 50 g of freeze dried powder.

Active and surfactant ingredient components were dissolved together in alcohol in order to ensure that the surfactant component was intimately mixed with the oxprenolol component prior to entry to freeze drying. In the case of addition of other inert ingredients, such as the sugar alcohol mannitol, and the surfactant sodium lauryl sulphate, the alcoholic solution containing drug/surfactant was added in a 60% ratio to water containing mannitol prior to freeze drying. The following types and quantities of materials were used:

(a) Freeze Drying with Surfactant Mixture 1

| | Concentration | | |
|---|---|---|---|
| Chemical/Grade | (percent w/w) | | |
| Atenolol HCl | 50 | 60 | 70 |
| Colfosceril palmitate | 35 | 28 | 21 |
| Phosphatidyl glycerol | 15 | 12 | 9 |

(b) Freeze Drying with Surfactant Mixture 2

| | Concentration | | |
|---|---|---|---|
| Chemical/Grade | (percent w/w) | | |
| Atenolol HCl | 50 | 60 | 70 |
| Phosphatidyl choline | 35 | 28 | 21 |
| Phosphatidyl glycerol | 15 | 12 | 9 |

(c) Freeze Drying with Surfactant and Sugar Alcohol Mixtures

| | Concentration | | |
|---|---|---|---|
| Chemical/Grade | (percent w/w) | | |
| Atenolol HCl | 40 | 50 | 60 |
| Colfosceril palmitate | 28 | 21 | 14 |
| Phosphatidyl glycerol | 12 | 9 | 6 |
| Mannitol | 20 | 20 | 20 |

(d) Freeze Drying with Non-Ionic and Anionic Surfactant Mixtures

| | Concentration | | |
|---|---|---|---|
| Chemical/Grade | (percent w/w) | | |
| Atenolol Base | 45 | 55 | 65 |
| Colfosceril palmitate | 35 | 28 | 21 |
| Phosphatidyl glycerol | 15 | 12 | 9 |
| Sodium lauryl sulphate | 5 | 5 | 5 |

(e) Freeze Drying with Surfactant and Sugar Alcohol Mixtures

| | Concentration | | |
|---|---|---|---|
| Chemical/Grade | (percent w/w) | | |
| Atenolol base | 40 | 50 | 60 |
| Colfosceril palmitate | 28 | 21 | 14 |
| Phosphatidyl glycerol | 12 | 9 | 6 |
| Sodium lauryl sulphate | 2 | 3 | 4 |
| Mannitol | 18 | 17 | 16 |

Freeze drying was carried out using an Edwards High Vacuum laboratory freeze drier operated under normal conditions.

Following freeze drying, matrix particles were milled to less than 10 μm particle size and dry blended with other inert ingredients to produce an organoleptically acceptable powder for administration.

### EXAMPLE 4

### Fast Dissolving Paracetamol for Peroral Administration—Dissolution Results

Dissolution tests were performed on two spray dried paracetamol formulations, batch numbers 025#21/01 & 025#21/02 using a Type 2 Dissolution apparatus. The samples were analysed by UV characterisation.

The two formulations had the following make up:

| | Batch No. | |
|---|---|---|
| | 025#21/01 | 025#21/02 |
| Amount drug in formulation (% w/w) | 50 | 80 |
| Amount mannitol in formulation (% w/w) | 49 | 19 |
| Amount SDS in formulation (% w/w) | 1 | 1 |
| Dissolution sample weight (mg) | 1000 | 625 |
| Nominal dose per sample (mg) | 500 | 500 |

The details of the methods used are summarised below:

| | |
|---|---|
| Method File name: | RUN 1: 50 & 80% SD APAP -0.1M HCl |
| | RUN 2: 50 & 80% SD APAP - Water |
| Apparatus: | Pharmatest Type 2 Dissolution Apparatus |
| Media: | RUN 1: 0.1M HCl |
| | RUN 2: Water |
| Volume per vessel: | 900 ml |
| Vessel temperature: | 37 ± 0.5° C. |
| Agitator: | Paddle |
| Speed: | 50 rpm |
| Analytical wavelength: | 243 nm |
| Cell pathlength: | 1 mm (online) & 10 mm (offline) |

The results are set out in the table below, with the release of paracetamol (%) measured at various time points (minutes):

| | 0.1M HCl | | | | | |
|---|---|---|---|---|---|---|
| | 025#21/01 | | | 025#21/02 | | |
| | Vessel | Vessel | | Vessel | | Vessel |
| Timepoint | 1 | 2 | Vessel 3 | 4 | Vessel 5 | 6 |
| 0 | 0.00 | 0.00 | 0.00 | 0.00 | 0.00 | 0.00 |
| 5 | 78.70 | 91.56 | 92.16 | 84.04 | 90.67 | 96.00 |
| 10 | 99.38 | 99.60 | 100.97 | 97.63 | 100.67 | 101.93 |
| 15 | 102.57 | 100.15 | 100.84 | 99.87 | 100.67 | 100.81 |
| 20 | 101.37 | 100.90 | 100.06 | 102.73 | 100.87 | 101.70 |
| 30 | 100.81 | 100.20 | 99.64 | 100.22 | 100.86 | 101.56 |

| | Water | | | | | |
|---|---|---|---|---|---|---|
| | 025#21/01 | | | 025#21/02 | | |
| | | Vessel | Vessel | | Vessel | Vessel |
| Timepoint | Vessel 1 | 2 | 3 | Vessel 4 | 5 | 6 |
| 0 | 0.00 | 0.00 | 0.00 | 0.00 | 0.00 | 0.00 |
| 5 | 98.17 | 92.23 | 84.97 | 85.76 | 91.52 | 86.74 |
| 10 | 97.75 | 98.96 | 97.05 | 94.59 | 98.14 | 93.40 |
| 15 | 98.79 | 99.85 | 101.09 | 99.34 | 100.56 | 97.07 |
| 30 | 100.31 | 102.19 | 101.09 | 100.31 | 100.83 | 99.84 |

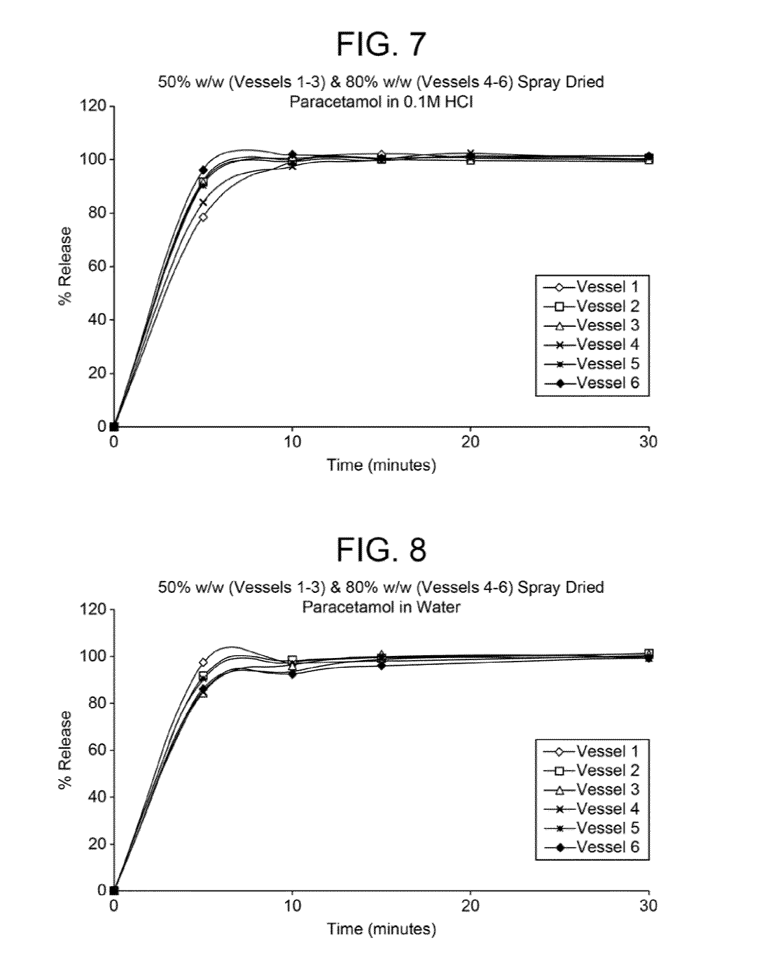

These results are shown in the graphs of FIGS. 7 and 8. FIG. 7 shows the results for the 50% w/w (vessels 1-3) and 80% w/w (vessels 4-6) spray dried paracetamol in 0.1M HCl. FIG. 8 shows the results for the 50% w/w (vessels 1-3) and 80% w/w (vessels 4-6) spray dried paracetamol in water.

From these results, it can been seen that Batches 025#21/01 & 025#21/02 behaved similarly in 0.1M HCl and Water. The “in 0.1M HCl” samples released between 79-96% at 5 minutes and all samples released above 95% within 10 minutes. The “in water” samples released between 85-98% at 5 minutes and all samples released above 95% within 15 minutes. During dissolution test it was noticed that powder initially dispersed onto the surface of the media but rapidly dropped to the bottom of the vessel.

### EXAMPLE 5

### Paclitaxel for Transmucosal Administration Encapsulation Efficiency for Spray Dried Submicron Particles

Submicron particles containing paclitaxel and a biopolymer, polylactideglycolide (PLGA) were prepared using an emulsifier. Selection of a particular emulsifier, whether synthetic polymers, e.g. polyvinyl alcohol (PVA), or natural macromolecules such as phospholipids and cholesterol can be used to control submicron drug size and size distribution, drug encapsulation efficiency, morphological properties, mucosal spreading and in vitro release profiles of the drug. The drug is dissolved in an organic solvent with the biopolymer, methylene chloride (also known as dichloromethane). The resulting solution is subsequently added to distilled water containing any water soluble inert ingredients, such as polymers and sugar alcohols. The emulsifier can be added either in the oil or in the water phase, depending on its solubility properties. The resulting emulsion is then spray-dried.

As can be seen from the figures in the table below, in this example, compared with PVA, phospholipids result in a smaller size, a narrower size distribution, and a higher encapsulation efficiency (EE). Phospholipids were also found to be more effective emulsifiers than PVA. In this example, the amount of phospholipid needed was only 1/40 (by weight) of the PVA to achieve the same emulsifying effect.

| Emulsifier | EE (%) | Mean Size (nm) |
|---|---|---|
| PVA (2 wt %) | 40.2 | 973.5 + 41.0 |
| PVA (4 wt %) | 22.9 | 801.0 + 38.0 |
| DPPC (0.05 wt %) | 44.9 | 571.0 + 89.0 |
| DPPC (0.1 wt %) | 34.0 | 633.0 + 134.0 |

Unsaturated lipids have been found not to be effective in emulsification. Also among various saturated lipids, those with shorter chains yield better results. For example, DDPC can result in a smaller size, a narrower size distribution and a much higher EE, as shown by the figures in the table below.

| Emulsifier | EE (%) | Mean Size (nm) |
|---|---|---|
| DDPC (10:0) | 87.2 | 426.7 + 10.6 |
| DPPC (16:0) | 44.9 | 571.0 + 89.0 |
| DSPC (18:0) | 39.8 | 829.1 + 30.2 |

### EXAMPLE 6

### Pig Skin Experiments & Data

The analytical method used in this study is described in the table below:

| | |
|---|---|
| HPLC System | Waters Alliance 2695 Separations Module plus Autosampler |
| | Waters detector 2487, Empower Prop Software |
| Column | Phenomenex HyperClone 5 μ 250 × 4.6 mm BDS C18 |
| Guard Column | 1 cm Generic C18, Hichrom Ltd |
| Detection | 282 nm |
| Sample Temp | 8 ± 5° C. |
| Column Temp | Ambient |
| Flow Rate | 1 ml/min |
| Mobile Phase | Phosphate/dibutylamine buffer was prepared as follows: |
| | 0.970 g of dibutylamine, 0.735 g of phosphoric acid and 2.93 g |
| | of sodium dihydrogen phosphate dissolved in 750 ml of |
| | water, pH adjusted to 6.5 with strong sodium hydroxide |
| | solution (10M) and made up to 1 L with water deionised |
| | water. |
| | Composition: 90% phosphate/dibutylamine buffer, 10% |
| | acetonitrile |
| Injection Volume | 10 μl |
| Run Time | 10 min |
| Autosampler Vials | Borosilicate glass vials |
| SS Retention Time | ~7 min |

The receiver fluid chosen for the investigation was 10% ethanol in PBS, as this had shown a maximum solubility of 1.933 mg/ml and it was thought that it would not limit the permeation of the drug into the receiver fluid. The stability of the raw drug (sumatriptan) in the receiver fluid was determined when stored at 4° C., 25° C., 37° C. and −20° C. over a period of 48 hours.

| Concentration | Temperature | Peak |
|---|---|---|
| (μg/ml) | (° C.) | area |
| 50 | −20 | 467385 |
| | 4 | 469462 |
| | 25 | 470115 |
| | 37 | 473418 |
| 1 | −20 | 9514 |
| | 4 | 9366 |
| | 25 | 9415 |
| | 37 | 7094 |

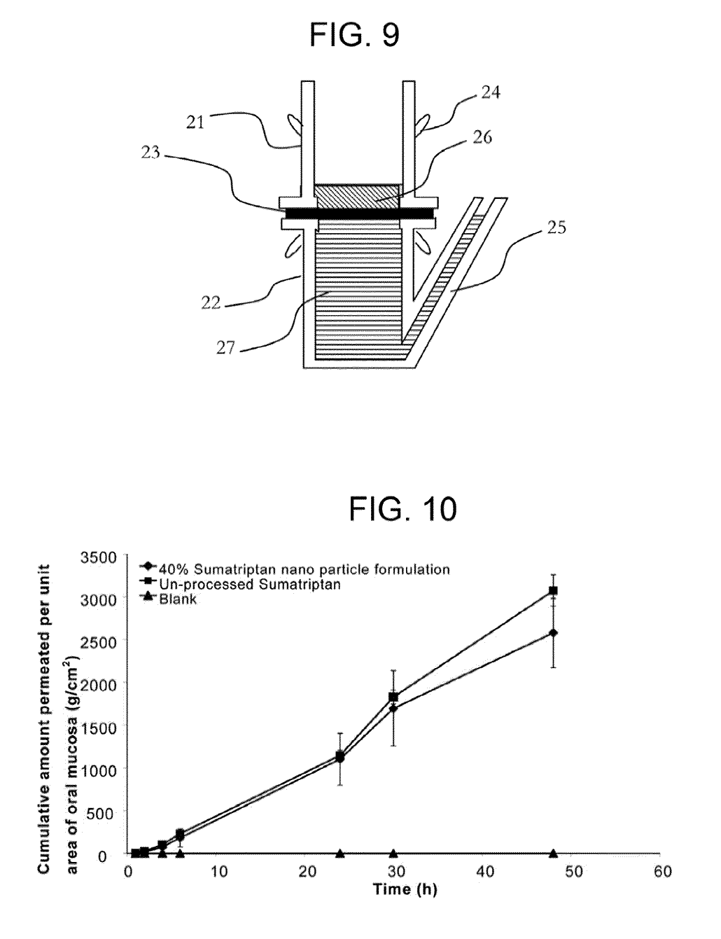

A Franz cell was set up as shown in FIG. 9, using an oral pig mucosa 23 mounted in between the donor compartment 21 and receiver compartment 22. A permeation study was performed in order to investigate over what period of time a quantifiable amount of drug could be detected in the receiver fluid.

Individually calibrated Franz cells with an average surface area and volume of approximately 0.6 cm² and 2 ml respectively were employed to determine the permeation of sumatriptan from the submicron particle formulation. The oral pig mucosa was mounted between two halves of the Franz cell with the mucosal side facing the donor compartment. The receptor compartment 22, having a clamp attachment lug 24, was filled with receiver fluid 27 (10% ethanol in PBS), stirred constantly with a PTFE-coated magnetic follower driven by submersible magnetic stirrer and maintained at 37° C. in a water bath. Approximately 5 mg of each formulation 26 was placed into the donor compartment 21 and the donor compartment was covered with Parafilm® throughout the study. Following the application of the drug formulation, the receiver fluid 27 (200 μl) was removed from the receiver compartment 22 via the sampling arm 25 after, e.g. 1, 2, 4, 6, 24 and 48 hours and analysed via HPLC. Each removed sample was replaced by an equal volume of fresh pre-warmed (37° C.) receiver fluid.

At the end of the experiment, a mass balance investigation was performed as follows:

A. Surface drug (S), the surface of the oral mucosa containing each formulation was wiped carefully using a sequence of dry and wet cotton buds. Collectively, the cotton buds from each Franz cell were immersed into glass vials containing 5 ml of receiver fluid. The vials were placed in an oscillating shaker and left overnight. An aliquot of 1 ml of the sample was then removed and analysed via HPLC.
B. The oral mucosa was placed into a glass vial containing 5 ml of receiver fluid, and placed on a shaker at room temperature overnight. An aliquot of 1 ml of the sample was then removed and analysed via HPLC.
C. Franz cell receptor compartment interface (joining section with donor) was swabbed with cotton buds. Collectively, the cotton buds from each Franz cell were immersed into a glass vial containing 5 ml receiver fluid. The vials were then placed in an oscillating shaker and left overnight. An aliquot of 1 ml of the sample was then removed and analysed via HPLC.
D. Franz cell donor compartment interface (joining section with receptor) and internal face of the donor compartment was swabbed with cotton buds. Collectively, the cotton buds from each Franz cell were immersed into a glass vial containing 5 ml receiver fluid. The vials were then placed in an oscillating shaker and left overnight. An aliquot of 1 ml of the sample was then removed and analysed via HPLC.

This experiment was carried out with the submicron sumatriptan formulation (a remade batch corresponding to Example 1A, batch 25#37/01) (n=6), pure unprocessed sumatriptan (n=3) and blank (n=2), i.e. cells with no formulation applied. The results are shown in FIG. 10, which shows the mean cumulative amount of sumatriptan permeated per unit area (μg/cm²) as a function of time (h). Test Item 1 is the 40% sumatriptan submicron particle formulation (approximately 5 mg applied to the surface of the mucosa, n=6±SD), Test Item 2 is the unprocessed sumatriptan raw material (approximately 5 mg applied to the surface of the mucosa, n=3±SD) and the Blank is no Test Item applied (n=2).

The average amount of sumatriptan recovered from each matrix following mass balance investigation is shown below:

| | 40% | | | |
|---|---|---|---|---|
| | Sumatriptan submicron | | | |
| | particle formulation | | Unprocessed sumatriptan | |
| | | % recovered | | % recovered |
| | Sumatriptan | compared | Sumatriptan | compared |
| | recovered | to what | recovered | to what |
| | (mg) | was applied | (mg) | was applied |
| Total amount | 1444.52 | 63.47 | 1924.07 | 29.86 |
| in receiver | | | | |
| fluid | | | | |
| Mucosa | 301.99 | 13.27 | 522.03 | 8.10 |
| Surface | 186.53 | 8.20 | 3178.59 | 49.33 |
| Receptor | 11.18 | 0.49 | 18.25 | 0.28 |
| Donor | 3.54 | 0.16 | 95.93 | 1.49 |
| Plunger | 129.87 | 5.71 | 345.81 | 5.37 |

The total amount recovered compared to the theoretical recovery based on the weight of each formulation applied.

| | Mean | SD | % CV |
|---|---|---|---|
| 40% Sumatriptan submicron | 91.28 | 5.40 | 5.98 |
| particle formulation | | | |
| Unprocessed Sumatriptan | 94.43 | 2.71 | 2.87 |

### EXAMPLE 7

### Paracetamol Particles from Emulsions

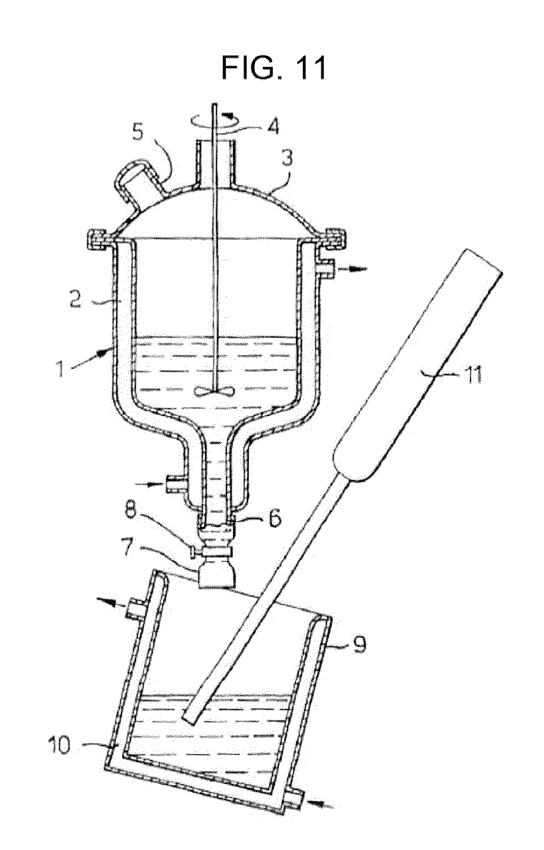

An emulsion of the following composition was prepared using the apparatus shown in FIG. 11.

### Oil Phase:

| | |
|---|---|
| Soybean oil | 36.6% by weight |
| Sorbitan monooleate | 5.3% by weight |
| Poly (oxyethylene) hydrogenated castor oil | 1.1% by weight |

### Aqueous Phase:

| | |
|---|---|
| Deionised water (Milli-Q) | 55.3% by weight |
| Paracetamol (Sigma) | 1.7% by weight |

The apparatus shown in FIG. 11 comprises an upper vessel of volume 500 cm³ for holding the disperse phase. The upper vessel 1 has a temperature controlled water jacket 2, a lid 3 having a central port for stirrer shaft 4 and a port 5 for addition of material. In the bottom of the upper vessel 1 is outlet 6 over which is fitted a length of PVC tubing 7 having a clip 8 to act as a flow control.

Fixed below the outlet 6 is a lower vessel 9 for holding the continuous phase initially and the emulsion when formed. The lower vessel 9 also has a water jacket 10. Homogeniser 11 is arranged to stir the contents of the lower vessel 9.

The water was placed in the upper vessel 1 and heated to 70° C. The paracetamol was then added to the water via the small neck in the upper vessel lid and the solution was stirred until all the paracetamol dissolved.

The oil phase was added to the lower vessel 9 and heated to 70° C. The homogeniser 11 was started at 16000 rpm and the clip 8 on the PVC tubing 7 was loosened to allow a slow drip of the aqueous phase into the oil. The clip was gradually loosened over time so that the flow was increased as the emulsion began to form. Once all the aqueous phase had been added the speed of homogenisation was increased to maximum of 24000 rpm for a few minutes. The emulsion was then cooled at a rate of 20° C. per hour, with low speed homogenisation. The particles were isolated by filtering the emulsion under vacuum and were washed with a little cold water. Alternatively, dry particles were prepared by freeze drying, and spray drying.

### EXAMPLE 8

### BSA Particles from Microemulsions

Particles of a biological macromolecule, bovine serum albumin (Molecular weight=67,000) are prepared by the method described below, using the following materials:

1) An aqueous buffered solution of bovine serum albumin (BSA), prepared by dissolving 60 mg/cm³ of the BSA in a 50 mM sodium acetate (NaAC) buffer at a pH of 5.0.
2) A saturated solution of the precipitating substance-ammonium sulphate, prepared in an aqueous 50 mM sodium acetate buffer at a pH of 5.0.
3) An oily isopropyl myristate.
4) A surfactant, dioctyl sulphosuccinate sodium salt.

Deionized water is used in the preparation of all aqueous solutions.

Two separate microemulsions are prepared at ambient temperature in 30 cm³ vials. For each microemulsion, 5 g of the surfactant and 10 g of the oil are combined and rapidly stirred. In preparing each water-in-oil microemulsion, equal volumes of the aqueous solution of BSA and the ammonium sulphate solution are added dropwise to the rapidly stirred surfactant-oil mixtures.

The amount of aqueous solutions mixed in each microemulsion are chosen to obtain the desired molar ratio of the water to surfactants, R=[water/surfactant] for example R=25. The radius of the dispersive water pool in the continuous oil phase may be changed by varying R. R is preferably in the range of 20 to 56.

After formation of two microemulsions, the two water-in-oil microemulsions are rapidly mixed together in a 100 ml vial. Due to the exchange process which subsequently occurs between droplets (described below), the mixing of the microemulsions results in size-controlled crystallites of the bovine serum albumin by precipitation of the protein within the water droplets.

It is possible to control the crystal form, and shape and size of the protein particles by varying the concentration of the solution of ammonium sulphate. Also, for proteins with a temperature-dependent solubility, the temperature of the vials can be controlled to combine precipitation with crystallisation of the protein. For example, if the temperature of the mixed microemulsion is maintained at between 8 and 17° C. spherical agglomerates are formed, whereas if the temperature is maintained between 18 and 37° C., non-spherical crystals are formed. The particles are isolated by filtration. The concentration of surfactant is then reduced by washing the particles in an excess of the ammonium sulphate solution. Dry particles were prepared by freeze drying and spray drying.

### Dynamics of Exchange Process

Since the precipitation in a mixed water-in-oil microemulsion is confined within the dispersed water droplets, a necessary step prior to precipitation is the transfer of the reactants into the same droplet. The readiness with which that process occurs in any given system is determined by the inter-micellar exchange rate constant, kₑₓ and the diffusion-controlled droplet collision, k_{diff}. These rates of exchange and diffusion are a function of each particular water-in-oil emulsion system, and can be controlled by varying the temperature, nature and/or amount of surfactants and by adding additive substances.

### EXAMPLE 9

### Paracetamol Particles from Emulsion Crystallisation

This example illustrates the use of an emulsion crystallisation technique, which utilises the temperature solubility dependence of paracetamol to crystallise paracetamol particles/crystals having a low polydispersity.

The following liquid phases are used:

1. A saturated solution of paracetamol, prepared by dissolving 65 g of paracetamol in 100 g of deionized water at 70° C. When saturated, the solution is filtered into the reservoir vessel. To avoid any unwanted precipitation/crystallisation the solution is maintained at ˜70° C. which is a few degrees above the saturation point of the solution.
2. An oil phase containing soybean oil and surfactants polyoxyethylene castor oil derivative and sorbitan monooleate. 41 g of soybean oil, 1.5 g of polyoxyethylene caster oil derivative and 7.5 g of sorbitan monooleate are combined in a temperature controlled jacketed vessel, and forcefully stirred at 70° C.

150 g of the saturated paracetamol solution is added dropwise, via a capillary tube, at a controlled flow rate into the stirred oil. Upon formation of the emulsion, under intense stirring, paracetamol crystallisation is induced within the water droplets by lowering the temperature of the emulsion. Control of crystallisation can be achieved both by varying the temperature drop between the saturated and crystallisation temperatures, and by using a predetermined temperature ramp in the crystallisation vessel thereby controlling the nucleation and subsequent growth of the paracetamol particles/crystals.

The amount of paracetamol solution mixed in the emulsion is chosen to obtain the desired molar ratio of water to surfactants, R=[water]/[surfactant], for example, R=25. The radius of the dispersive water pool that is, the radius of the droplets, may be changed by varying R. Preferably, R is in the range of from 25 to 56.

The crystals of paracetamol are separated from the emulsion by filtration under vacuum and washing with a suitable solvent. Alternatively, the volatile components of the emulsion may be removed by distillation. Alternatively dry particles were prepared by freeze drying and spray drying.

### EXAMPLE 10

### Dexamethasone Particles from Emulsions (I)

This example illustrates the use of an oil-in-water emulsion of the following composition:

| | |
|---|---|
| Castor oil (saturated with dexamethasone) | 15% by weight |
| Sorbitan monooleate | 4.25% by weight |
| Polyoxyethylene-(20)-sorbitan monooleate | 0.75% by weight |
| Water | 80% by weight |

The surfactants (sorbitan monooleate and polyoxyethylene-(20)-sorbitan monooleate) are dissolved in the castor oil/dexamethasone at 70° C. The water is heated to 70° C. and the castor oil/surfactant mixture added with stirring at about 700 rpm. The emulsion is homogenised for 1 minute using a high shear mixture and then cooled to room temperature under continued stirring.

The formation of solid particles of the dexamethasone may be induced either by adding a water soluble precipitant substance, by reducing the temperature of by changing the pH. Alternatively, dry particles were prepared by freeze drying and spray drying.

### EXAMPLE 11

### Dexamethasone Particles from Emulsions (II)

This example illustrates the use of a surfactant-free emulsion of the following composition:

| | |
|---|---|
| Soybean oil (saturated with dexamethasone) | 20% by weight |
| Water | 75% by weight |
| Poly (acrylic acid) | 5% by weight |

The soybean oil and the water are separately heated to 75° C. and combined under stirring at 700 rpm. The emulsion is homogenised with a high shear mixer for 1 minute and then the poly (acrylic acid) is dispersed in the emulsion with stirring. Dry particles were prepared by freeze drying and spray drying.

### EXAMPLE 12

### Dexamethasone Particles from Multiple Emulsions

This example illustrates the use of a multiple emulsion, specifically a water-in-oil-in-water emulsion.

In a first stage a primary emulsion of the following composition is prepared.

### Primary Emulsion

### Water-in-Oil

| | | |
|---|---|---|
| A | Glyceryl monostearate | 3% |
| | Sorbitan monooleate | 3% |
| | Soybean oil | 29% |
| B | Water (saturated with | 61% |
| | terbutaline or ipratropium) | |
| | NaCl | 4% |

The soybean oil and the surfactants are mixed together to form mixture A which is heated to 75° C. The sodium chloride is dissolved in the water to give solution B which is also heated to 75° C. Solution B is added to mixture A whilst stirring at 700 rpm. The resulting primary emulsion is homogenised on a high shear mixer for 1 minute and is maintained at 75° C. whilst being stirred at 500 rpm.

A multiple emulsion of the following composition is then prepared. Dry particles can be prepared by freeze drying, spray drying or any other suitable drying method.

### Secondary Emulsion

### Water (1) in Oil in Water (2)

| | | |
|---|---|---|
| C | Primary emulsion | 60% |
| D | Poloxamer (POE/POP block co-polymer) | 2% |
| | Water | 15% |
| E | NaCl | 2% |
| F | Water | 20% |
| | Poly (acrylic acid) | 0.2% |

The poloxamer is dissolved in water at 5° C. to make solution D which is maintained at 5° C. The sodium chloride (E) and the primary emulsion are then added to solution D whilst stirring at 700 rpm to form an emulsion. As the primary emulsion cools on contact with solution D, crystallization of the terbutaline or ipratropium occurs. Solution F is then prepared by adding the poly (acrylic acid) to water until a homogenous gel is formed. F is then added in small portions to the emulsion whilst stirring at 400 rpm. Stirring at 300 rpm is continued until F is completely dispersed.

During the formation of the solid particles the oil may act as a semi permeable membrane and controls the rate of diffusion between the water droplets and the continuous phase. It is also possible to use an oil-soluble active substance which will dissolve in the soybean oil, such as dexamethasone. In that case, crystallisation may be induced from the inside of the oil droplet or from outside. Dry particles can be prepared by freeze drying, spray drying or any other suitable drying method.

All references including patent and patent applications referred to in this application are incorporated herein by reference to the fullest extent possible. Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A composition for transmucosal delivery of a therapeutically active agent, comprising submicron particles comprising the active agent, wherein the active agent is sparingly soluble or insoluble in water.

2. A method of treatment of a subject which comprises transmucosal delivery of a composition comprising a therapeutically active agent, comprising submicron particles comprising the active agent, wherein the active agent is sparingly soluble or insoluble in water.

3. A composition as claimed in claim 1, wherein the therapeutically active agent is in base form which is sparingly soluble or insoluble in water.

4-8. (canceled)

9. A composition as claimed in claim 1, wherein the therapeutically active agent is in acid form which is sparingly soluble or insoluble in water.

10. A composition as claimed in claim 1 claims, comprising the active agent in crystalline form.

11. A composition as claimed in claim 1, wherein the submicron particles comprise the active agent in amorphous form which is sparingly soluble or insoluble in water.

12. A composition as claimed in claim 1, wherein the submicron particles are capable of mucosal adhesion.

13. A composition as claimed in claim 1, wherein the submicron particles are capable of persistence at the mucosal surface for not less than 2 minutes.

14. A composition as claimed in claim 1, wherein the submicron particles are capable of spreading over an area of the mucosal surface equivalent to not less than 1.5 times the area over which the particles are first applied.

15. A composition as claimed claim 1, wherein the majority of the submicron particles have a diameter of between 100 nm and 10 μm.

16. (canceled)

17. A composition as claimed in claim 1, wherein the active agent has a solubility of 1 part (by weight) drug in no less than 30 parts (by volume) water at 25° C.

18. A composition as claimed in claim 1, wherein the submicron particles consist of one or more active agents.

19. A composition as claimed in claim 1, wherein the submicron particles comprise one or more active agents and one or more inert ingredients.

20. A composition as claimed in claim 1, wherein at least 1% of the administered dose of the active agent is delivered by pre-gastric transmucosal absorption.

21. (canceled)

22. A composition as claimed in claim 1, wherein at least 15% of the administered dose of the active agent is delivered by pre-gastric transmucosal absorption.

23. A composition as claimed in claim 1, wherein the submicron particles are dispersed within one or more inert materials which form a matrix.

24. A composition as claimed in claim 23, wherein the matrix material is in the form at least one particle containing submicron active agent particles, the matrix particle having a diameter of at least 1 μm.

25. A composition as claimed in claim 23, wherein the submicron particles are dispersed amongst particles of inert material which rapidly dissolves or disperses in an aqueous environment.

26. (canceled)

27. A composition as claimed in claim 25, wherein the inert material is selected from one or more of: water, other aqueous media (e.g. water-ethanol mixtures and isotonic water-glycerol mixtures) or non-aqueous media leading to residual levels in a pharmaceutical product suitable for administration to humans or animals; surfactants, including non-ionic surfactants, anionic, cationic and amphoteric surfactants such as polysorbates (e.g. Tweens), and polyoxyethylene sorbitan fatty acid esters, sorbitan esters (e.g. Spans, sorbitan monostearate), including sorbitan laurate, sorbitan oleate, sorbitan palmitate, sorbitan sesquioleate, sorbitan stearate, sorbitan trioleate, sorbitan tristearate, sucrose esters, poloxamers (e.g. Pluronics) including poloxamer 188, poloxamer 407 and poloxalene, polyoxyl castor oils, polyoxyl hydrogenated castor oils, propylene glycol diacetate, propylene glycol laurate, propylene glycol dilaurate, propylene glycol monopalmitostearate, quillaia, diacetylated monoglycerides, diethylene glycol monopalmitostearate, p-di-isobutyl-phenoxypolyethoxyethanol, ethylene glycol monostearate, self-emulsifying glyceryl monostearate, macrogol cetostearyl ethers, cetomacrogol, polyoxyethylenes, polyethylene glycols, polyoxyl 20 cetostearyl ether, macrogol 15 hydroxystearate, macrogol laurel ethers, laureth 4, lauromacrogol 400, macrogol monomethyl ethers, macrogol oleyl ethers, menfegol, mono- and di-glycerides, nonoxinols, octoxinols, glyceryl distearate, glyceryl monolinoleate, glyceryl mono-oleate, tyloxapol, free fatty acids (e.g. oleic acid, palmitic acid, stearic acid, behenic acid, erucic acid) and their salts and esters (e.g. sodium stearate, magnesium stearate, aluminium monostearate, calcium stearate, zinc stearate, sodium cetostearyl sulphate, sodium oleate, sodium stearyl fumarate, sodium tetradecyl sulphate, soft soap, sulphated castor oil, glyceryl behenate), phospholipids and phospholipid-containing materials, including phosphatidylcholine, lecithin, colfosceril palmitate, phosphatidyl glycerol, Lucinactant, animal lung extracts and modified animal lung extracts; sodium lauryl sulphate and docusate sodium, benzalkonium chloride, cetrimide and nonylphenols, and other emulsifiers (including polymeric materials); soluble small molecules including amino acids (e.g. taurine, aspartame) and especially bioadhesive materials, including sugars, sugar alcohols, dextrates, dextrins, dextrans and hydrating agents, especially urea; and soluble large molecules, especially biodegradable polymers capable of dissolving or dispersing relatively rapidly, including natural and semi-synthetic macromolecules such as phospholipids and especially those that can aid adhesion to and/or spreading across mucosal surfaces (e.g. phosphatidyl choline, lyso-phosphatidyl choline, colfosceril palmitate, phosphatidyl glycerol and mixtures of such materials including with e.g. tyloxapol, cetyl alcohol, free fatty acids), vitamins, natural oils including orange, lemon, bergamot, anise; alcohols, including menthol and cetyl alcohol and cholesterol, natural polymers such as xanthan, guar and alginates, synthetic polymers such as PVP and PVA, semi-synthetic polymers such as cellulose derivatives (e.g. HPMC and HPC) and starch derivatives.

28. A composition as claimed in claim 1, further comprising a solvent, wherein the solvent is an alcohol or oil.

29-31. (canceled)

32. A composition as claimed in claim 31, wherein at least about 5% of the dose of active agent enters the systemic circulation within 15 to 30 minutes following administration.

33. A composition as claimed in claim 32, wherein an appropriate pharmacodynamic measure shows therapeutic activity within 15 to 30 minutes following administration.

34. (canceled)

35. A composition as claimed in claim 1, wherein the active agent is a drug that exhibits high “first-pass” metabolism, a drug that shows “food effects”, a drug that exhibits variable or poor absorption due to GI disturbances, a drug that undergoes chemical or enzymatic degradation in the stomach or intestines, a drug that has a principle site of action in the central nervous system, a drug that is intended to provide rapid or acute treatment of symptoms, an acid or GI labile drug, a drug that is taken into the body via a lipid uptake mechanism, a drug that is in a poorly soluble base form, a BCS Class II drug, a BCS Class III drug and/or a BCS Class IV drug.

36. A composition comprising the base chemical form of a therapeutically active agent in submicron physical form, for rapid delivery of the active agent both into the systemic circulation and across the blood-brain-barrier.

37-39. (canceled)

40. A composition as claimed in claim 1, wherein the composition is a loose powder, a capsule containing a loose powder or powder compressed into a solid dosage form.

41. (canceled)

42. A composition for transmucosal delivery of a therapeutically active agent, wherein the active agent is sumatriptan and is sparingly soluble or insoluble in water.

43. (canceled)

44. A composition as claimed in claim 42, wherein the sumatriptan is in base form which is sparingly soluble or insoluble in water.

45. A composition as claimed in claim 42, comprising the sumatriptan in crystalline form.

46. A composition as claimed in claim 42 wherein the submicron particles comprise the sumatriptan in amorphous form which is sparingly soluble or insoluble in water.

47. A composition as claimed in claim 42 wherein the submicron particles are capable of mucosal adhesion.

48. A composition as claimed in claim 42, wherein the submicron particles are capable of persistence at the mucosal surface for not less than 2 minutes.

49. A composition as claimed in claim 42, wherein the submicron particles are capable of spreading over an area of the mucosal surface equivalent to not less than 1.5 times the area over which the particles are first applied.

50. A composition as claimed in claim 42, wherein the majority of the submicron particles have a diameter of between 100 nm and 10 μm.

51. (canceled)

52. A composition as claimed in claim 42, wherein the sumatriptan has a solubility of 1 part (by weight) drug in no less than 30 parts (by volume) water at 25° C.

53. A composition as claimed in claim 42, wherein the submicron particles comprise or consist of sumatriptan and one or more other active agents.

54. A composition as claimed in claim 42, wherein the submicron particles comprise or consist of sumatriptan and one or more inert ingredients.

55. A composition as claimed in claim 42, wherein at least 1% of the administered dose of sumatriptan is delivered by pre-gastric transmucosal absorption.

56. A composition as claimed in claim 55, wherein at least 5% or 15% of the administered dose of sumatriptan is delivered by pre-Gastric transmucosal absorption.

57. A composition as claimed in claim 42, wherein the submicron particles are dispersed within one or more inert materials which form a matrix.

58. A composition as claimed in claim 57, wherein the matrix material is in the form at least one particles containing submicron active agent particles, the matrix particle having a diameter of at least 1 μm.

59. A composition as claimed in claim 57, wherein the submicron particles are dispersed amongst particles of inert material which rapidly dissolves or disperses in an aqueous environment.

60. (canceled)

61. A composition as claimed in claim 59, wherein the inert material is selected from one or more of: water, other aqueous media (e.g. water-ethanol mixtures and isotonic water-glycerol mixtures) or non-aqueous media leading to residual levels in a pharmaceutical product suitable for administration to humans or animals; surfactants, including non-ionic surfactants, anionic, cationic and amphoteric surfactants such as polysorbates (e.g. Tweens), and polyoxyethylene sorbitan fatty acid esters, sorbitan esters (e.g. Spans, sorbitan monostearate), including sorbitan laurate, sorbitan oleate, sorbitan palmitate, sorbitan sesquioleate, sorbitan stearate, sorbitan trioleate, sorbitan tristearate, sucrose esters, poloxamers (e.g. Pluronics) including poloxamer 188, poloxamer 407 and poloxalene, polyoxyl castor oils, polyoxyl hydrogenated castor oils, propylene glycol diacetate, propylene glycol laurate, propylene glycol dilaurate, propylene glycol monopalmitostearate, quillaia, diacetylated monoglycerides, diethylene glycol monopalmitostearate, p-di-isobutyl-phenoxypolyethoxyethanol, ethylene glycol monostearate, self-emulsifying glyceryl monostearate, macrogol cetostearyl ethers, cetomacrogol, polyoxyethylenes, polyethylene glycols, polyoxyl 20 cetostearyl ether, macrogol 15 hydroxystearate, macrogol laurel ethers, laureth 4, lauromacrogol 400, macrogol monomethyl ethers, macrogol oleyl ethers, menfegol, mono- and di-glycerides, nonoxinols, octoxinols, glyceryl distearate, glyceryl monolinoleate, glyceryl mono-oleate, tyloxapol, free fatty acids (e.g. oleic acid, palmitic acid, stearic acid, behenic acid, erucic acid) and their salts and esters (e.g. sodium stearate, magnesium stearate, aluminium monostearate, calcium stearate, zinc stearate, sodium cetostearyl sulphate, sodium oleate, sodium stearyl fumarate, sodium tetradecyl sulphate, soft soap, sulphated castor oil, glyceryl behenate), phospholipids and phospholipid-containing materials, including phosphatidylcholine, colfosceril palmitate, phosphatidyl glycerol, Lucinactant, animal lung extracts and modified animal lung extracts; sodium lauryl sulphate and docusate sodium, benzalkonium chloride, cetrimide and nonylphenols, and other emulsifiers (including polymeric materials); soluble small molecules including amino acids (e.g. taurine, aspartame) and especially bioadhesive materials, including sugars, sugar alcohols, dextrates, dextrins, dextrans and hydrating agents, especially urea; and soluble large molecules, especially biodegradable polymers capable of dissolving or dispersing relatively rapidly, including natural and semi-synthetic macromolecules such as phospholipids and especially those that can aid adhesion to and/or spreading across mucosal surfaces (e.g. phosphatidyl choline, lyso-phosphatidyl choline, colfosceril palmitate, phosphatidyl glycerol and mixtures of such materials including with e.g. tyloxapol, cetyl alcohol, free fatty acids), vitamins, natural oils including orange, lemon, bergamot, anise; alcohols, including menthol and cetyl alcohol and cholesterol, natural polymers such as xanthan, guar and alginates, synthetic polymers such as PVP and PVA, semi-synthetic polymers such as cellulose derivatives (e.g. HPMC and HPC) and starch derivatives.

62. A composition as claimed in claim 42, further comprising a solvent, wherein the solvent is an alcohol or oil.

63. A composition as claimed in claim 42, wherein at least 15% of the dose of active agent is delivered without “first pass” metabolism, without being affected by “food effects” or by GI disturbances.

64-65. (canceled)

66. A composition as claimed in claim 65, wherein at least about 5% of the dose of sumatriptan enters the systemic circulation within 15 to 30 minutes following administration.

67. A composition as claimed in claim 66, wherein an appropriate pharmacodynamic measure shows therapeutic activity within 15 to 30 minutes following administration.

68-71. (canceled)

72. A composition as claimed in claim 42, wherein the composition is a loose powder, a capsule containing a loose powder or powder compressed into a solid dosage form.

73-82. (canceled)
